SEQUENCE LISTING (7 sequences)

SEQ ID NO: 1 - protein, 639 aa, Artificial Sequence
  Description of Artificial Sequence Synthetic polypeptide
MSQITIQCCLIASESTRQKLWKLMAHLNTPLINELLQQLSKHPDFEKWRKNGKLPSTVVNQLCQPLKTDPSFTGQPSRLYLSAIHVVDYIYKSWLAIQKRLQQQLDGKIRWLEMLNSDAELIEISGCSLEAIRTKAAEILAIATPDSDVAAPLTKTGKAKKSKKSSASNPDRSLSHKLFDAYQETDDILSRSAISYLLRNGCKLNDKEEDLEKFAKRRRKVEIQIQRLTDKLTSRIPKGRDLTNAKWLETLFTATTTVPEDNVEAKRWQDILLTRSSSVPFPLIFETNEDLVWSKNEKGRLCVHFNGLSDLTFEVYCDRRQLHWFKRFLEDQQTKRKSKNQHSSGLFTLRNGRLAWQEGEGKGEPWQINRLTLYCCVDNRLWSAEGTEQVRQEKEEEITKFITKMNEKSDLSETQQAFIKRKESTLTRINNSFERPSQFLYQGQSHILVGVSLGLEKPATVAVVDAIAGKVLAYRSIKQLLGDNYELLNRQRRQQQYLSHERHKAQKSFSPNQFGTSELGQYVDRLLAKEIIAIAQTHKAGSIVLPKLGDMREIVQSEIQAIAEEKFPGYVEGQQKYAKQYRVNVHGWSHSRLIQSIQSKAAQIGIVIEEGKQPIRGSPQDKAKELALSAYNLRLARRS

SEQ ID NO: 2 - protein, 584 aa, Artificial Sequence
  Description of Artificial Sequence Synthetic polypeptide
MNSQQNPDLAVHTSAIPTEGLQEESDKPLERNVIVTALSEEAQLKLEVIQSLLEKSDRTTYGQKLKEAAEKLSVSVRTVQRLVKKWEQDGVVGFTQTGRADKGRHRIGEFWENFILKTYREGNKGSKRMTPKQVALRVQAKARELEDSKPPNYRTVLRVLAPVLEKQEKAKSIRSPGWRGTTLSVKTREGKDLSVDYSNHVWQCDHTRVDVLLVDQHGQLLSRPWLTTVIDTYSRCIMGINLGFDAPSSVVVALALRHAILPKQYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYLVDRYNQSIDARMGDQTRFERWEAGLPTVPVPIPERDLDICLMKQSRRTVQRGGCLQFQNLMYQGEYLAGYAGETVNLRFDPRDITTILVYRQENNQEVFLTRAHAQGLETEQLALDEAEAASRRLRTAGKTISNQSLLQEVVDRDALVATKKSRKERQKLEQAVLRSAGVDESKTESLSSQVVEPDEVESTAEINSQYEDMEVWDYEQLREEYGF

SEQ ID NO: 3 - protein, 276 aa, Artificial Sequence
  Description of Artificial Sequence Synthetic polypeptide
MTEASAIAKQLGGVKPDDEWLQAEIARLKGKSIVPLQQVKTLHDWLDGKRKARQSCRVVGESRTGKTVACDAYRYRHKPHQEVGRPPIVPVVYIQPPQKCGSKDLFKEMIEYLKFRATKGTVSDFRGRAMEVLKGCEVEMLIIDEADRLKPETFAEVRDIYDKLGIAVVLVGTDRLEAVIKRDEQVYNRFRACHRFGKLSGKDFQDTVQAWEDRVLKLPVSSNLTSKDMLRILTLATEGYIGRLDEILRETAIRSLSKGFKKIDKAVLQEVAKEYK

SEQ ID NO: 4 - protein, 167 aa, Artificial Sequence
  Description of Artificial Sequence Synthetic polypeptide
MTAPDVKPWLFIIEPYPGESLSHFLGRFRRANHLSAAGLGNLAGIGAVVARWERFHFNPRPSQQELEAIASVVEVDADRLAQMLPPLGVGMQHEPIRLCGACYAVTPCHQIEWQFKEMGGCNRHNLRLLSKCPKCEDRFKIPALWEYGLCGRCRTPFAEMAECQKRI

SEQ ID NO: 5 - RNA, 254 nt, Artificial Sequence
  Description of Artificial Sequence Synthetic polynucleotide
  modified_base (232)..(254): a, c, u, g, unknown or other
auaauaaauagcgccgcaguucaugcugcuugcagccugugaacuguguuaaaugaggguuaguuugacuguagcaauacagucuugcuuucugacccuaguagcugcucacccugaugcugcugucuucggacaggauaggugcgcucccagcaauaagggcgcggauguacugcuguaguggcuacugaaucacccccgaucaagggggaacccuaaaaggguugaaagnnnnnnnnnnnnnnnnnnnnnnn

SEQ ID NO: 6 - DNA, 238 bp, Artificial Sequence
  Description of Artificial Sequence Synthetic polynucleotide
tgtacagtgactaattatatgtcgtcggtgacaaattgttgtcattgagccagactagttgtcgtcgtggcaaattaggtgtcgctcatttaatggtgacaaattaatgtcgctaagataatacactctgtaattatcatacagaacaattcaaacaagcggataaaaggacttgctttcaacccacccctaaatttaatacttactgaaacccaaaactaatctcagtttcaaatat

SEQ ID NO: 7 - DNA, 262 bp, Artificial Sequence
  Description of Artificial Sequence Synthetic polynucleotide
aatacataagttttttataaatacatattgttattctaggtgtagataaagcgacagtcaatttgtcattatgaaaatacacaaaagcttttttctatttaaaaaagcgacaactaatttgtcactattacggataacgacatttattttgtcaccatgaagaagtgatcctaattttgtgaaaacgctataagatatactgtacaagcattttagcaatgacattaatttgtcacgacgacaaataaaaagtcactgtaca

(19) United States
(12) Patent Application Publication
(10) Pub. No.: US 2024/0301371 A1
(43) Pub. Date: Sep. 12, 2024

(54) CRISPR-ASSOCIATED TRANSPOSON SYSTEMS AND METHODS OF USING SAME

(71) Applicant: Arbor Biotechnologies, Inc., Cambridge, MA (US)

(72) Inventors: Kyle Edward Watters, Cambridge, MA (US); Noah Michael Jakimo, Cambridge, MA (US); Chad David Torgerson, Cambridge, MA (US)

(73) Assignee: Arbor Biotechnologies, Inc., Cambridge, MA (US)

(21) Appl. No.: 18/274,697

(22) PCT Filed: Jan. 28, 2022
(86) PCT No.: PCT/IB2022/050783

§ 371 Date: Jul. 27, 2023

Related U.S. Application Data
(60) Provisional application No. 63/142,990, filed on Jan. 28, 2021.

(51) Int. Cl. C12N 9/12; C12N 9/22; C12N 15/11; C12N 15/70; C12R 1/19

(52) U.S. Cl. CPC C12N 9/1241; C12N 9/22; C12N 15/111; C12N 15/70; C12N 2310/20; C12R 2001/19

(57) ABSTRACT

The present disclosure relates to systems, compositions and methods for modifying target nucleic acid sequences.

### RELATED APPLICATIONS

This application claims priority to U.S. Provisional Application No. 63/142,990, filed on Jan. 28, 2021. The entire contents of the foregoing priority application are incorporated by reference herein.

### SEQUENCE LISTING

The instant application contains a Sequence Listing which has been submitted electronically in ASCII format and is hereby incorporated by reference in its entirety. Said ASCII copy, created on Jan. 26, 2022, is named A112029_1020 WO_(0010_7)_SL.txt and is 16,715 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

Described herein are recombinant nucleic acid compositions and recombinant nucleic acid targeting systems for sequence-specific modification of a target sequence, as well as methods of using recombinant nucleic acid targeting systems.

In one aspect, the disclosure provides a recombinant nucleic acid comprising a first promoter operably linked to a first polynucleotide and a second promoter operably linked to a second polynucleotide. The first polynucleotide comprises a nucleic acid sequence encoding at least one Clustered Interspaced Short Palindromic Repeat (CRISPR)-associated transposase protein, or functional fragment thereof, and a nucleic acid sequence encoding a CRISPR associated (Cas) protein. The second polynucleotide comprises a nucleic acid sequence encoding a guide RNA (gRNA) that is capable of hybridizing with a target sequence.

In another aspect, the disclosure provides a recombinant nucleic acid comprising a first promoter operably linked to a first polynucleotide and a second promoter operably linked to a second polynucleotide, wherein the first polynucleotide comprises a nucleic acid sequence encoding a TniA protein, or functional fragment thereof, a nucleic acid sequence encoding a TniB protein, or functional fragment thereof, and a nucleic acid sequence encoding a TniQ protein, or functional fragment thereof, and a nucleic acid sequence encoding a CRISPR associated (Cas) protein, wherein the Cas protein comprises an amino acid sequence set forth in SEQ ID NO: 1; wherein the second polynucleotide comprises a nucleic acid sequence encoding a guide RNA (gRNA), wherein the gRNA is capable of hybridizing with a target sequence.

In yet another aspect, the disclosure provides a recombinant nucleic acid comprising a first promoter operably linked to a first polynucleotide and a second promoter operably linked to a second polynucleotide, wherein the first polynucleotide comprises a nucleic acid sequence encoding a TniA protein, or functional fragment thereof, a nucleic acid sequence encoding a TniB protein, or functional fragment thereof, and a nucleic acid sequence encoding a TniQ protein, or functional fragment thereof, and a nucleic acid sequence encoding a CRISPR associated (Cas) protein, wherein the Cas protein comprises an amino acid sequence that is at least 95% identical to an amino acid sequence set forth in SEQ ID NO: 1; wherein the second polynucleotide comprises a nucleic acid sequence encoding a guide RNA (gRNA), wherein the gRNA is capable of hybridizing with a target sequence.

In one embodiment, the recombinant nucleic acid comprises at least one CRISPR-associated transposase protein, or functional fragment thereof, comprising one or more proteins selected from the group consisting of a TniA protein, a TniB protein, and a TniQ protein. In another embodiment, the at least one CRISPR-associated transposase protein, or functional fragment thereof, comprises two or more proteins selected from the group consisting of a TniA protein, a TniB protein, and a TniQ protein. In yet another embodiment, the at least one CRISPR-associated transposase protein, or functional fragment thereof, comprises TniA protein, a TniB protein, and a TniQ protein. In certain embodiments described above, the TniA protein comprises an amino acid sequence that is at least 95% identical to an amino acid sequence set forth in SEQ ID NO: 2. In certain embodiments described above, the TniA protein comprises an amino acid sequence set forth in SEQ ID NO: 2. In certain embodiments described above, the TniB protein comprises an amino acid sequence that is at least 95% identical to an amino acid sequence set forth in SEQ ID NO: 3. In certain embodiments described above, the TniB protein comprises an amino acid sequence set forth in SEQ ID NO: 3. In certain embodiments described above, the TniQ protein comprises an amino acid sequence that is at least 95% identical to an amino acid sequence set forth in SEQ ID NO: 4. In certain embodiments described above, the TniQ protein comprises an amino acid sequence set forth in SEQ ID NO: 4.

In some embodiments, recombinant nucleic acid comprises the first polynucleotide that comprises a nucleic acid sequence encoding the TniA protein comprising an amino acid sequence as set forth in SEQ ID NO: 2, a nucleic acid sequence encoding the TniB protein comprising an amino acid sequence as set forth in SEQ ID NO: 3, and a nucleic acid sequence encoding the TniQ protein comprising an amino acid sequence as set forth in SEQ ID NO: 4.

In some embodiments, recombinant nucleic acid comprises the first polynucleotide that comprises a nucleic acid sequence encoding the TniA protein comprising an amino acid sequence that is at least 95% identical to an amino acid sequence set forth in SEQ ID NO: 2, a nucleic acid sequence encoding the TniB protein comprising an amino acid sequence that is at least 95% identical to an amino acid sequence set forth in SEQ ID NO: 3, and a nucleic acid sequence encoding the TniQ protein comprising an amino acid sequence that is at least 95% identical to an amino acid sequence set forth in SEQ ID NO: 4.

In some embodiments, the recombinant nucleic acid comprises a nucleic acid sequence encoding a Cas protein that is a Type V-K Cas protein. In some embodiments, the Type V-K Cas protein is Cas12k protein comprising an amino acid sequence that is at least 95% identical to an amino acid sequence as set forth in SEQ ID NO: 1. In specific embodiments, the Cas12k protein comprises an amino acid sequence as set forth in SEQ ID NO: 1.

In one embodiment, the recombinant nucleic acid comprises a first polynucleotide comprising a nucleic acid sequence encoding a TniA protein, or functional fragment thereof, a nucleic acid sequence encoding a TniB protein, or functional fragment thereof, and a nucleic acid sequence encoding a TniQ protein, or functional fragment thereof, and a nucleic acid sequence encoding a Cas protein (e.g., Cas12k protein) comprising an amino acid sequence as set forth in SEQ ID NO: 1. The recombinant nucleic acid further comprises a second polynucleotide comprising a nucleic acid sequence encoding a gRNA that is capable of hybridizing with a target sequence.

In some embodiments, the recombinant nucleic acid comprises a gRNA that is capable of complexing with the Cas protein (e.g., Cas12k protein) to form a Cas protein/gRNA ribonucleoprotein (RNP) complex. In some embodiments, the gRNA comprises a CRISPR/Cas system associated RNA (crRNA) sequence. In certain embodiments, the gRNA is a single guide RNA further comprising a trans-activating CRISPR/Cas system RNA (tracrRNA) sequence. In some embodiments, the gRNA comprises a nucleotide sequence as set forth in SEQ ID NO: 5.

In one aspect, the disclosure provides a vector comprising the recombinant nucleic acids herein. In another aspect, the disclosure provides a bacterial cell comprising the vector described herein.

In one aspect, the disclosure provides a recombinant nucleic acid targeting system for sequence-specific modification of a target sequence. The system comprises at least one CRISPR-associated transposase protein or a polynucleotide encoding the at least one CRISPR-associated transposase protein, a Cas protein (e.g., Cas12k protein) or a polynucleotide encoding the Cas protein; and a guide RNA (gRNA) or a polynucleotide encoding the gRNA. In some embodiments, the recombinant nucleic acid targeting system comprises a gRNA that is capable of complexing with the Cas protein to form a Cas protein/gRNA RNP complex.

In one embodiment, the recombinant nucleic acid targeting system comprises at least one CRISPR-associated transposase protein, or functional fragment thereof, comprising one or more proteins selected from the group consisting of a TniA protein, a TniB protein, and a TniQ protein. In another embodiment, the at least one CRISPR-associated transposase protein, or functional fragment thereof, comprises two or more proteins selected from the group consisting of a TniA protein, a TniB protein, and a TniQ protein. In yet another embodiment, the at least one CRISPR-associated transposase protein, or functional fragment thereof, comprises TniA protein, a TniB protein, and a TniQ protein. In certain embodiments described above, the TniA protein comprises an amino acid sequence that is at least 95% identical to an amino acid sequence set forth in SEQ ID NO: 2. In certain embodiments described above, the TniA protein comprises an amino acid sequence set forth in SEQ ID NO: 2. In certain embodiments described above, the TniB protein comprises an amino acid sequence that is at least 95% identical to an amino acid sequence set forth in SEQ ID NO: 3. In certain embodiments described above, the TniB protein comprises an amino acid sequence set forth in SEQ ID NO: 3. In certain embodiments described above, the TniQ protein comprises an amino acid sequence that is at least 95% identical to an amino acid sequence set forth in SEQ ID NO: 4. In certain embodiments described above, the TniQ protein comprises an amino acid sequence set forth in SEQ ID NO: 4.

In some embodiments, recombinant nucleic acid targeting system comprises the first polynucleotide that comprises a nucleic acid sequence encoding the TniA protein comprising an amino acid sequence that is at least 95% identical to an amino acid set forth in SEQ ID NO: 2, a nucleic acid sequence encoding the TniB protein comprising an amino acid sequence that is at least 95% identical to an amino acid set forth in SEQ ID NO: 3, and a nucleic acid sequence encoding the TniQ protein comprising an amino acid sequence that is at least 95% identical to an amino acid set forth in SEQ ID NO: 4. In other embodiments, recombinant nucleic acid targeting system comprises the first polynucleotide that comprises a nucleic acid sequence encoding the TniA protein comprising an amino acid sequence as set forth in SEQ ID NO: 2, a nucleic acid sequence encoding the TniB protein comprising an amino acid sequence as set forth in SEQ ID NO: 3, and a nucleic acid sequence encoding the TniQ protein comprising an amino acid sequence as set forth in SEQ ID NO: 4.

In some embodiments, the recombinant nucleic acid targeting system comprises a nucleic acid sequence encoding a Cas protein that is a Type V-K Cas protein. In some embodiments, the Type V-K Cas protein is Cas12k protein comprising an amino acid sequence that is at least 95% identical to an amino acid sequence as set forth in SEQ ID NO: 1. In specific embodiments, the Cas12k protein comprises an amino acid sequence as set forth in SEQ ID NO: 1.

In one embodiment, the recombinant nucleic acid targeting system for sequence-specific modification of a target sequence comprises a TniA protein, a TniB protein, and a TniQ protein, or a polynucleotide encoding the TniA protein, the TniB protein, and the TniQ protein, a Cas protein comprising an amino acid sequence as set forth in SEQ ID NO: 1 or a polynucleotide encoding the Cas protein comprising an amino acid sequence as set forth in SEQ ID NO: 1 and a gRNA or a polynucleotide encoding the gRNA, wherein the gRNA is capable of complexing with the Cas protein to form a gRNA-Cas protein complex.

In some embodiments, the recombinant nucleic acid targeting system comprises a gRNA comprising a CRISPR/Cas system associated RNA (crRNA) sequence. In certain embodiments, the gRNA is a single guide RNA further comprising a trans-activating CRISPR/Cas system RNA (tracrRNA) sequence. In some embodiments, the gRNA comprises a nucleotide sequence as set forth in SEQ ID NO: 5.

In some embodiments, the recombinant nucleic acid targeting system further comprises a target polynucleotide. The target polynucleotide comprises (i) a target sequence capable of hybridizing to the gRNA and (ii) a protospacer-adjacent motif (PAM) sequence. In certain embodiments, the PAM comprises the nucleotide sequence 5′-GTN-3′, 5′-NGTN-3′, or 5′-GGTN-3′. In certain embodiments, the PAM comprises the nucleotide sequence 5′-GGTT-3′. In certain embodiments, the PAM comprises the nucleotide sequences 5′-GTT-3′, 5′-GTA-3′, 5′-GTC-3′, or 5′-GTG-3′. In certain embodiments, the PAM comprises 5′-GGTA-3′, 5′-GGTC-3′, or 5′-GGTG-3′. In a particular embodiment, the PAM comprises a nucleotide sequence as set forth in 5′-GGTT-3′.

In some embodiments, the recombinant nucleic acid targeting system further comprises a donor polynucleotide. The donor polynucleotide comprises a payload sequence for insertion into the target polynucleotide. In some embodiments, the donor polynucleotide further comprises a nucleic acid sequence encoding a transposon left end (TE-L) and a nucleic acid sequence encoding a transposon right end (TE-R). In certain embodiments, the TE-L comprises a nucleic acid sequence that is at least 95% identical to a nucleic acid sequence set forth in SEQ ID NO: 6. In certain embodiments, the TE-L comprises a nucleic acid sequence as set forth in SEQ ID NO: 6. In certain embodiments, the TE-R comprises a nucleic acid sequence that is at least 95% identical to a nucleic acid sequence set forth in SEQ ID NO: 7. In certain embodiments, the TE-R comprises a nucleic acid sequence as set forth in SEQ ID NO: 7.

In some embodiments, the recombinant nucleic acid targeting system comprises a TniA protein comprising an amino acid sequence that is at least 95% identical to an amino acid sequence set forth in SEQ ID NO: 2 and a donor polynucleotide, wherein the donor polynucleotide comprises a payload sequence for insertion into the target sequence, a nucleic acid sequence encoding a transposon left end (TE-L) that is at least 95% identical to a nucleic acid sequence set forth in SEQ ID NO: 6, and a nucleic acid sequence encoding a transposon right end (TE-R) that is at least 95% identical to a nucleic acid sequence set forth in SEQ ID NO: 7. In certain embodiments, the recombinant nucleic acid targeting system further comprises a Cas protein (e.g., Cas12k protein) comprising an amino acid sequence that is at least 95% identical to an amino acid sequence set forth in SEQ ID NO: 1 or a polynucleotide encoding the Cas protein, wherein the Cas protein comprises an amino acid sequence that is at least 95% identical to an amino acid sequence set forth in SEQ ID NO: 1 and a guide RNA (gRNA) or a polynucleotide encoding the gRNA, wherein the gRNA is capable of complexing with the Cas protein to form a gRNA-Cas protein complex. In certain embodiments, the recombinant nucleic acid targeting system further comprises one or more of a TniB protein and a TniQ protein.

In certain embodiments, the recombinant nucleic targeting system comprises at least one of the Cas protein (e.g., Cas12k protein), the TniA protein, the TniB protein, and the TniQ protein as purified protein.

In one aspect, the disclosure provides a bacterial cell comprising the recombinant nucleic acid targeting system described herein.

In one aspect, the disclosure provides a method for modifying a target polynucleotide in a bacterial cell. The method comprises introducing into to the cell a first, second and third recombinant nucleic acids. The first recombinant nucleic acid comprises a polynucleotide encoding at least one CRISPR-associated transposase protein, or functional fragment thereof, a polynucleotide encoding a Cas protein (e.g., Cas12k protein); and a polynucleotide encoding a gRNA. The second recombinant nucleic acid comprises a target polynucleotide comprising a target sequence capable of hybridizing to the gRNA and a PAM sequence. The third recombinant nucleic acid comprises a donor polynucleotide that comprises a payload sequence for insertion into the target polynucleotide.

In some embodiments of the method described herein, the gRNA is capable of complexing with the Cas protein to form a Cas protein/gRNA RNP complex.

In one embodiment of the method for modifying a target polynucleotide in a bacterial cell, the method comprises introducing into to the cell a first recombinant nucleic acid comprising a polynucleotide encoding a TniA protein, or functional fragment thereof, a polynucleotide encoding a TniB protein, or functional fragment thereof, and a polynucleotide encoding a TniQ protein, or functional fragment thereof, a polynucleotide encoding a Cas protein comprising an amino acid sequence as set forth in SEQ ID NO: 1 and a polynucleotide encoding a gRNA that is capable of complexing with the Cas protein to form a gRNA-Cas protein complex. In the embodiment described above, the method further comprises introducing into to the cell a second recombinant nucleic acid comprising a target polynucleotide that comprises a target sequence capable of hybridizing to the gRNA and a PAM sequence. The method further comprises introducing into to the cell a third recombinant nucleic acid comprising a donor polynucleotide that comprises a payload sequence for insertion into the target polynucleotide.

In some embodiments of the method described herein, the recombinant nucleic acid targeting system further comprises a donor polynucleotide. The donor polynucleotide comprises a payload sequence for insertion into the target polynucleotide. In some embodiments, the donor polynucleotide further comprises a nucleic acid sequence encoding a TE-L and a nucleic acid sequence encoding a TE-R. In certain embodiments, the TE-L comprises a nucleic acid sequence as set forth in SEQ ID NO: 6. In certain embodiments, the TE-R comprises a nucleic acid sequence as set forth in SEQ ID NO: 7.

In one embodiment of the method, the recombinant nucleic acid comprises a polynucleotide comprising at least one CRISPR-associated transposase protein, or functional fragment thereof. In some embodiments, the polynucleotide encodes a TniA protein, or functional fragment thereof, a TniB protein, or functional fragment thereof, or a TniQ protein, or functional fragment thereof. In another embodiment, the at least one CRISPR-associated transposase protein, or functional fragment thereof, comprises two or more proteins selected from the group consisting of a TniA protein, a TniB protein, and a TniQ protein. In yet another embodiment, the at least one CRISPR-associated transposase protein, or functional fragment thereof, comprises TniA protein, a TniB protein, and a TniQ protein. In certain embodiments described above, the TniA protein comprises an amino acid sequence that is at least 95% identical to an amino acid sequence set forth in SEQ ID NO: 2. In certain embodiments described above, the TniB protein comprises an amino acid sequence that is at least 95% identical to an amino acid sequence set forth in SEQ ID NO: 3. In certain embodiments described above, the TniQ protein comprises an amino acid sequence that is at least 95% identical to an amino acid sequence set forth in SEQ ID NO: 4. In some embodiments of the method, the TniA protein comprises an amino acid sequence that is at least 95% identical to an amino acid sequence set forth in SEQ ID NO: 2, the TniB protein comprises an amino acid sequence that is at least 95% identical to an amino acid sequence set forth in SEQ ID NO: 3, and the TniQ protein comprises an amino acid sequence that is at least 95% identical to an amino acid sequence set forth in SEQ ID NO: 4. In some embodiments of the method, the TniA protein comprises an amino acid sequence as set forth in SEQ ID NO: 2, the TniB protein comprises an amino acid sequence as set forth in SEQ ID NO: 3, and the TniQ protein comprises an amino acid sequence as set forth in SEQ ID NO: 4.

In some embodiments of the method, the PAM comprises the nucleotide sequence 5′-GTN-3′, 5′-NGTN-3′, or 5′-GGTN-3′. In certain embodiments, the PAM comprises the nucleotide sequence 5′-GGTT-3′. In certain embodiments, the PAM comprises the nucleotide sequences 5′-GTT-3′, 5′-GTA-3′, 5′-GTC-3′, or 5′-GTG-3′. In certain embodiments, the PAM comprises 5′-GGTA-3′, 5′-GGTC-3′, or 5′-GGTG-3′. In a particular embodiment, the PAM comprises a nucleotide sequence as set forth in 5′-GGTT-3′.

In some embodiments of the method, the bacterial cell is Escherichia coli.

### BRIEF DESCRIPTION OF THE DRAWINGS

FIG. 1A depicts the structure of the pEffector plasmid A2 with coding regions for TniA, TniB, TniQ, Cas12k, a sgRNA scaffold, and an ampicillin resistance protein (AmpR). FIG. 1B depicts the structure of the pDonor plasmid B2 with a coding region for a payload sequence, which includes a kanamycin resistance gene, and the sequences of left (TE-L) and right (TE-R) transposon ends. FIG. 1C depicts the structure of the pTarget plasmid C2 with a protospacer adjacent motif (PAM) sequence and a coding region for a target sequence.

FIG. 2 shows pEffector plasmid A2-mediated CRISPR-associated transposase events for the insertion of the pDonor plasmid B2 payload sequence into the pTarget plasmid C2. The x- and y-axes represent the alignment position to the pTarget plasmid C2 and the pDonor plasmid B2, respectively, while the histograms in the vertical and horizontal axes display the number of sequencing reads in one of the paired-end reads aligning to the pDonor plasmid B2 or the pTarget plasmid C2, respectively.

### DETAILED DESCRIPTION

The present disclosure relates to recombinant nucleic acid compositions and recombinant nucleic acid targeting systems for sequence-specific modification of a target sequence. The disclosure also provides methods for modifying a target polynucleotide in a bacterial cell. The compositions and methods described herein comprise polynucleotides encoding one or more Clustered Interspaced Short Palindromic Repeat (CRISPR)-associated transposase proteins, or functional fragments thereof, one or more components of a sequence-specific nucleotide binding protein (e.g., a Cas protein), and a guide molecule (e.g. guide RNA molecule). The compositions and methods described herein further comprise a target polynucleotide comprising a target sequence capable of hybridizing to the gRNA and a donor polynucleotide comprising a payload sequence for insertion into the target polynucleotide.

### I. Definitions

Unless otherwise defined, all terms used in the present disclosure have the meaning as commonly understood by one of ordinary skill in the art. By means of further guidance, term definitions are included to better appreciate the teachings of the present disclosure.

As used herein, the term “about” or “approximately”, when referring to a measurable value such as a parameter, an amount, and the like, is meant to encompass variations of +/−10% or less, preferably +/−5% or less, and more preferably +/−1% or less of and from the specified value, insofar such variations are appropriate to perform in the present disclosure.

As used herein, the term “donor polynucleotide” is a polynucleotide molecule that includes a payload sequence capable of being inserted into a target nucleic acid sequence using a CRISPR-associated transposase, or a method, as described herein.

As used herein, the term “effector complex” refers to a complex having at least one protein that carries out an enzymatic activity or that binds to a target site on a nucleic acid specified by a guide RNA.

As used herein, the term “encoding” or “coding for” refers to a nucleic acid sequence (i.e., DNA) that is transcribed (and optionally translated) when placed under the control of an appropriate regulatory sequence(s).

As used herein, the term “hybridization” refers to a reaction in which one or more polynucleotides interact to form a complex that is stabilized via hydrogen bonding between the bases of the residues of the polynucleotides.

As used herein, the term “nucleic acid targeting system” refers to transcripts and other elements involved in the expression of, or that otherwise directs the activity of, a CRISPR-Cas-based system (e.g., a CRISPR-associated transposase system), which may include nucleotide sequences encoding a CRISPR-associated transposase system.

The term “operably linked”, as used herein, refers to a nucleic acid sequence (or nucleic acid sequences) of interest that is linked to a regulatory element(s) in a manner that allows for expression of the nucleotide sequence (or nucleotide sequences) of interest. The term “regulatory element” is intended to include promoters, ribosomal binding sites (RBSs), and other expression control elements.

As used herein, the term “payload sequence” refers to a nucleic acid sequence (e.g., a DNA sequence or an RNA sequence) of interest that is capable of being integrated into a target sequence. The payload sequence may be a sequence that is endogenous or exogenous to a cell (e.g., a bacterial cell). Non-limiting examples of a payload sequence include a DNA sequence, a RNA sequence encoding a protein, and a non-coding RNA sequence (e.g., a microRNA).

As used herein, “promoter” refers to a DNA sequence located upstream of, or at the 5′ end of, a transcription initiation site (or protein-coding region) of a gene and that is involved in recognition and binding of an RNA polymerase and other proteins (trans-acting transcription factors) to initiate transcription.

As used herein, the term “protospacer adjacent motif” or “PAM” refers to a DNA sequence adjacent to a target sequence to which a complex comprising an effector complex and an RNA guide binds. In some embodiments, a PAM is required for enzyme activity.

As used herein, the terms “guide RNA” or “gRNA” or “guide RNA sequence” refer to any RNA molecule that facilitates the targeting of a polypeptide described herein to a target nucleic acid sequence. For example, an RNA guide can be a molecule that recognizes (e.g., binds to) a target nucleic acid sequence. A guide RNA may be synthetically designed to be complementary to a specific nucleic acid sequence. In one aspect, a guide RNA provided herein comprises a CRISPR RNA (crRNA). In one aspect, a guide RNA provided herein comprises a CRISPR RNA (crRNA) complexed with a trans-activating CRISPR RNA (tracrRNA). In another aspect, a guide RNA provided herein comprises a single-chain guide RNA (sgRNA). In one aspect, a single-chain guide RNA provided herein comprises both a crRNA and a tracrRNA.

As used herein, the term “substantially identical” refers to a sequence, i.e., a polynucleotide sequence or a polypeptide sequence, that has a certain degree of identity to a reference sequence.

As used herein, the terms “target sequence”, “target nucleic acid”, “target nucleic acid sequence” and “target site” refers, interchangeably, to a nucleotide sequence modified by a CRISPR-associated transposase or by a method as described herein. In some embodiments, the target sequence is in a gene.

As used herein, the term “target polynucleotide” refers to a polynucleotide molecule that includes a target sequence capable of having inserted therein a payload sequence using a CRISPR-associated transposase or a method as described herein.

As used herein, the terms “trans-activating crRNA” and “tracrRNA” refer to any polynucleotide sequence that has sufficient complementarity with a crRNA sequence to hybridize and is involved in or required for the binding of a guide RNA to a target nucleic acid.

### II. Compositions and Systems

The present disclosure provides recombinant nucleic acid compositions and recombinant nucleic acid targeting systems for sequence-specific modification of a target sequence. In one aspect, the disclosure provides a recombinant nucleic acid comprising a first promoter operably linked to a first polynucleotide and a second promoter operably linked to a second polynucleotide. In some embodiments, the first polynucleotide comprises a nucleic acid sequence encoding at least one Clustered Interspaced Short Palindromic Repeat (CRISPR)-associated transposase protein, or functional fragment thereof, and a nucleic acid sequence encoding a CRISPR associated (Cas) protein. In some embodiments, the second polynucleotide comprises a nucleic acid sequence encoding a guide RNA (gRNA) capable of hybridizing with a target sequence. In another aspect, the present disclosure provides a recombinant nucleic acid targeting system for sequence-specific modification of a target sequence. In some embodiments, the nucleic acid targeting system comprises at least one CRISPR-associated transposase protein, or a polynucleotide encoding the at least one CRISPR-associated transposase protein, a CRISPR associated (Cas) protein (e.g., Cas12k protein), or a polynucleotide encoding the Cas protein, and a guide RNA (gRNA), or a polynucleotide encoding the gRNA. In another embodiment, the nucleic acid targeting systems (or the recombinant nucleic acids) provided herein comprise at least one, at least two, at least three, at least four, or at least five (or more) promoters operably linked to at least one, at least two, at least three, at least four, or at least five polynucleotides encoding at least one, at least two, at least three, at least four, or at least five (CRISPR)-associated transposase protein(s). In some embodiments, the nucleic acid targeting systems (or the recombinant nucleic acids) provided herein encode at least one, at least two, at least three, at least four, or at least five (or more) guide RNAs. In some embodiments, the nucleic acid targeting systems further comprise at least one nucleic acid sequence encoding a transposon left end (TE-L) and at least one nucleic acid sequence encoding a transposon right end (TE-R).

In some embodiments, the nucleic acid targeting systems further comprise at least one target sequence capable of hybridizing to at least one of the gRNAs and at least one protospacer-adjacent motif (PAM) sequence.

### A. CRISPR-Associated Transposases

The recombinant nucleic acid compositions and recombinant nucleic acid targeting systems described herein comprise at least one CRISPR-associated transposase protein, or functional fragment thereof. For example, in some embodiments, the disclosure provides a recombinant nucleic acid composition comprising a first polynucleotide encoding at least one CRISPR-associated transposase protein, or functional fragment thereof. In other embodiments, the disclosure provides a recombinant nucleic acid targeting system comprising at least one CRISPR-associated transposase protein, or a polynucleotide encoding the at least one CRISPR-associated transposase protein. The term “transposase” refers to an enzyme that is capable of forming a functional complex with a transposon end sequence(s) (i.e., nucleotide sequences at the distal ends of a transposon) and catalyzing the insertion or transposition of a transposon end-containing sequence into a single- or double-stranded target nucleic acid sequence (e.g., DNA). The term “CRISPR-associated transposase” refers to transposase enzymes and/or proteins that are associated with a CRISPR locus. Further, as used herein, the term “transposition” or the term “transposition reaction” refers to a reaction wherein a transposase inserts a donor polynucleotide sequence (e.g., a payload sequence of a donor polynucleotide) into or adjacent to a target site in a target polynucleotide. In some embodiments, the payload sequence of a donor polynucleotide contains transposon end sequences (e.g., a transposon right end (TE-R) sequence and a transposon left (TE-L) end sequence) or a secondary structure elements recognized by the transposase, wherein upon recognition, the transposase cleaves or introduces staggered breaks in a target polynucleotide into which the payload sequence of the donor polynucleotide sequence may be inserted.

Exemplary transposases include, but are not limited to, Tn transposases (e.g., Tn3, Tn5, Tn7, Tn10, Tn552, Tn903), prokaryotic transposases, and any transposases related to and/or derived from the transposases provided herein. In certain embodiments, a transposase related to and/or derived from a parent transposase may comprise a polypeptide, or functional fragment thereof, with at least about 50%, about 55%, about 60%, about 65%, about 70%, about 75%, about 80%, about 81%, about 82%, about 83%, about 84%, about 85%, about 86%, about 87%, about 88, about 89%, about 90%, about 91%, about 92%, about 93%, about 94%, about 95%, about 96%, about 97%, about 98%, about 99%, or about 99.5% or more amino acid sequence homology to a corresponding polypeptide, or functional fragment thereof, of the parent transposase. In some embodiments, the at least one CRISPR-associated transposase protein described herein comprises a complete transposon system (e.g., a Tn7 transposon system). In some embodiments, the at least one (CRISPR)-associated transposase protein provided herein comprises an amino acid sequence having at least about 50% sequence identity, at least about 55% sequence identity, at least about 60% sequence identity, at least about 65% sequence identity, at least about 70% sequence identity, at least about 75% sequence identity, at least about 80% sequence identity, at least about 81% sequence identity, at least about 82% sequence identity, at least about 83% sequence identity, at least about 84% sequence identity, at least about 85% sequence identity, at least about 86% sequence identity, at least about 87% sequence identity, at least about 88% sequence identity, at least about 89% sequence identity, at least about 90% sequence identity, at least about 91% sequence identity, at least about 92% sequence identity, at least about 93% sequence identity, at least about 94% identity, at least about 95% sequence identity, at least about 96% sequence identity, at least about 97% sequence identity, at least about 98% sequence identity, at least about 99% sequence identity (or more) to at least one sequence selected from SEQ ID NOs: 2-4, or a functional fragment thereof. In some embodiments, the at least two (CRISPR)-associated transposase proteins provided herein comprises an amino acid sequence having at least about 50% sequence identity, at least about 55% sequence identity, at least about 60% sequence identity, at least about 65% sequence identity, at least about 70% sequence identity, at least about 75% sequence identity, at least about 80% sequence identity, at least about 81% sequence identity, at least about 82% sequence identity, at least about 83% sequence identity, at least about 84% sequence identity, at least about 85% sequence identity, at least about 86% sequence identity, at least about 87% sequence identity, at least about 88% sequence identity, at least about 89% sequence identity, at least about 90% sequence identity, at least about 91% sequence identity, at least about 92% sequence identity, at least about 93% sequence identity, at least about 94% identity, at least about 95% sequence identity, at least about 96% sequence identity, at least about 97% sequence identity, at least about 98% sequence identity, at least about 99% sequence identity (or more) to at least one sequence selected from SEQ ID NOs: 2-4, or a functional fragment thereof. In some embodiments, the at least three (CRISPR)-associated transposase protein provided herein comprises an amino acid sequence having at least about 50% sequence identity, at least about 55% sequence identity, at least about 60% sequence identity, at least about 65% sequence identity, at least about 70% sequence identity, at least about 75% sequence identity, at least about 80% sequence identity, at least about 81% sequence identity, at least about 82% sequence identity, at least about 83% sequence identity, at least about 84% sequence identity, at least about 85% sequence identity, at least about 86% sequence identity, at least about 87% sequence identity, at least about 88% sequence identity, at least about 89% sequence identity, at least about 90% sequence identity, at least about 91% sequence identity, at least about 92% sequence identity, at least about 93% sequence identity, at least about 94% identity, at least about 95% sequence identity, at least about 96% sequence identity, at least about 97% sequence identity, at least about 98% sequence identity, at least about 99% sequence identity (or more) to at least one sequence selected from SEQ ID NOs: 2-4, or a functional fragment thereof. In certain preferred embodiments, the compositions and systems described herein comprise at least one protein selected from a TniA protein, a TniB protein, and a TniQ protein, or a functional fragment thereof. In other preferred embodiments, the compositions and systems described herein comprise at least two proteins selected from a TniA protein, a TniB protein, and a TniQ protein, or a functional fragment thereof. In yet other preferred embodiments, the compositions and systems described herein comprise a TniA protein, a TniB protein, and a TniQ protein, or a functional fragment thereof.

In certain embodiments, the at least one CRISPR-associated transposase protein(s) described herein, may provide functions including, but not limited to, target cleavage and polynucleotide insertion. In specific embodiments, the at least one CRISPR-associated transposase protein(s) do not provide target polynucleotide recognition, but provide target polynucleotide cleavage and insertion of a donor polynucleotide into the target sequence. In other embodiments, the at least one CRISPR-associated transposase protein(s) provided herein forms a complex with the Cas protein/gRNA complex that directs the at least one CRISPR-associated transposase protein(s) to a target sequence of a target polynucleotide, wherein the at least one CRISPR-associated transposase protein(s) introduces two single-stranded breaks in the target polynucleotide where it inserts a donor polynucleotide. In certain embodiments, the target polynucleotide sequence can be single-stranded or double-stranded DNA. In some embodiments, formation of a complex comprising the Cas protein/gRNA ribonucleoprotein (RNP) complex and at least one CRISPR-associated transposase protein(s) results in insertion of the donor polynucleotide in one or both strands in or near (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80 or more base pairs from) a target sequence of a target polynucleotide. In other embodiments, formation of a complex comprising the Cas protein/gRNA RNP complex and at least one CRISPR-associated transposase protein(s) results in insertion of the donor polynucleotide in one or both strands in or near (e.g., within 1-10 base pairs, 5-15 base pairs, 10-20 base pairs, 15-25 base pairs, 20-30 base pairs, 25-35 base pairs, 30-40 base pairs, 35-45 base pairs, 45-60 base pairs, 45-70 base pairs, 45-80 base pairs or more base pairs from) a target sequence of a target polynucleotide.

The compositions and systems described herein comprise a CRISPR-Cas system and at least one CRISPR associated transposase protein(s). In some embodiments, a recombinant nucleic acid comprising one or more transgenes is integrated at the target site.

### B. Cas Protein and Guide RNA System

The recombinant nucleic acid compositions and recombinant nucleic acid targeting systems described herein comprise a CRISPR associated (Cas) protein (e.g., Cas12k protein), or a polynucleotide encoding a Cas protein. In certain embodiments, the Cas protein may serve as the nucleotide binding component of the recombinant nucleic acid targeting system. In certain embodiments, the at least one CRISPR-associated transposase protein(s) associates with, or forms a complex with a CRISPR associated (Cas) protein. In a preferred embodiment, the CRISPR associated (Cas) protein directs the at least one CRISPR-associated transposase protein(s) to a target sequence of a target polynucleotide where the at least one CRISPR-associated transposase protein(s) facilitates insertion of a payload sequence of a donor polynucleotide into the target sequence of the target polynucleotide.

In certain other embodiments, the recombinant nucleic acid compositions and the recombinant nucleic acid targeting systems described herein comprise a CRISPR associated (Cas) protein (e.g., Cas12k protein) or a polynucleotide encoding the Cas protein and a guide RNA (gRNA) capable of hybridizing with a target sequence of a target polynucleotide. In preferred embodiments, the gRNA is capable of complexing with the Cas protein to form a gRNA-Cas protein complex. In certain other embodiments, the Cas protein and the gRNA comprise the basic unit of a CRISPR-Cas system. In other embodiments, the guide RNA comprises one or more small interfering CRISPR RNAs (crRNAs) of approximately 60-80 nt in length, each of which associate with a trans-activating CRISPR RNA (tracrRNA) to guide the Cas protein (e.g., Cas12k) to the target sequence. The resulting CRISPR/Cas effector complex recognizes and binds to homologous double-stranded DNA sequences known as protospacers in a target sequence (e.g., DNA). In some embodiments, a prerequisite for cleavage is the presence of a conserved protospacer-adjacent motif (PAM) downstream of the target sequence. In certain embodiments, the PAM comprises the nucleotide sequence 5′-GTN-3′, 5′-NGTN-3′, or 5′-GGTN-3′. In certain embodiments, the PAM comprises the nucleotide sequence 5′-GGTT-3′. In certain embodiments, the PAM comprises the nucleotide sequences 5′-GTT-3′, 5′-GTA-3′, 5′-GTC-3′, or 5′-GTG-3′. In certain embodiments, the PAM comprises 5′-GGTA-3′, 5′-GGTC-3′, or 5′-GGTG-3′.

There are two classes of CRISPR-Cas systems generally recognized by those skilled in the art, which are referred to as Classes 1 and 2. Classes 1 and 2 are recognized as comprising multi-component, or single-component Cas proteins. In one aspect of the disclosure, a preferred system for cleaving or binding a target sequence of a target polynucleotide is a Cas protein of a Class 2, Type V CRISPR-Cas system (a Type V Cas protein). In some embodiments, the Type V Cas protein is a Type V-K Cas protein. In other preferred embodiments, the Type V-K Cas protein is a Cas12k protein. In some embodiments, the Cas12k protein comprises an amino acid sequence as set forth in SEQ ID NO: 1.

In some embodiments, the recombinant nucleic acid described herein comprises a nucleic acid sequence encoding a CRISPR associated (Cas) protein comprising an amino acid sequence having least about 60%, at least about 65%, at least about 70%, at least about 75%, having at least about 80% sequence identity, at least about 81% sequence identity, at least about 82% sequence identity, at least about 83% sequence identity, at least about 84% sequence identity, at least about 85% sequence identity, at least about 86% sequence identity, at least about 87% sequence identity, at least about 88% sequence identity, at least about 89% sequence identity, at least about 90% sequence identity, at least about 91% sequence identity, at least about 92% sequence identity, at least about 93% sequence identity, at least about 94% identity, at least about 95% sequence identity, at least about 96% sequence identity, at least about 97% sequence identity, at least about 98% sequence identity, at least about 99% sequence identity (or more) to the amino acid sequence as set forth in SEQ ID NO: 1. In certain other embodiments, the recombinant nucleic acid described herein comprises a polynucleotide encoding a Cas protein, wherein the Cas protein comprises an amino acid sequence having about 100% sequence identity to the amino acid sequence of the Cas12k protein as set forth in SEQ ID NO: 1. The percent identity between two sequences (e.g., nucleic acid or amino acid sequences) can be determined manually by inspection of the two optimally aligned amino acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the recombinant nucleic acid targeting system described herein comprises a CRISPR associated (Cas) protein or a polynucleotide encoding the Cas protein comprising an amino acid sequence having least about 60%, at least about 65%, at least about 70%, at least about 75%, having at least about 80% sequence identity, at least about 81% sequence identity, at least about 82% sequence identity, at least about 83% sequence identity, at least about 84% sequence identity, at least about 85% sequence identity, at least about 86% sequence identity, at least about 87% sequence identity, at least about 88% sequence identity, at least about 89% sequence identity, at least about 90% sequence identity, at least about 91% sequence identity, at least about 92% sequence identity, at least about 93% sequence identity, at least about 94% identity, at least about 95% sequence identity, at least about 96% sequence identity, at least about 97% sequence identity, at least about 98% sequence identity, at least about 99% sequence identity (or more) to the amino acid sequence set forth in SEQ ID NO: 1. In certain other embodiments, the recombinant nucleic acid targeting system described herein comprises a CRISPR associated (Cas) protein or a polynucleotide encoding the Cas protein comprising an amino acid sequence having about 100% sequence identity to the amino acid sequence of the Cas12k protein set forth in SEQ ID NO: 1. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or two or more conservative amino acid substitutions.

In some embodiments, the recombinant nucleic acid targeting system comprises one or more purified protein components. For example, the system may include one or more of a purified TniA protein, a purified TniB protein, a purified TniQ protein, and a purified Cas protein (e.g., Cas12k protein). Proteins in the system can be purified by methods known in the art. In certain embodiments, the protein components may include a tag to assist in expression, folding, stability, isolation, detection, and the like. In some embodiments, the tag is positioned at the C-terminus of the protein. In other embodiments, the tag is positioned at the N-terminus of the protein. In other embodiments, the tag is positioned at an internal position within the protein. The proteins disclosed herein can be tagged by functional protein tags known in the art. For example, an N-terminal His-SUMO tag can be used.

In some embodiments, the biochemistry of the Cas protein (e.g., Cas12k protein) described herein is analyzed using one or more assays. In some embodiments, the biochemical characteristics of a Cas protein of the present disclosure are analyzed in vitro using a purified Cas protein incubated with a guide RNA (e.g., an sgRNA) and a target polynucleotide (e.g., DNA molecule), as described in Examples 1 and 2.

In certain other embodiments, the recombinant nucleic acid and the recombinant nucleic acid targeting system described herein comprise a guide RNA (gRNA) capable of hybridizing with a Cas protein to form a gRNA-Cas protein complex. For example, in some embodiments, the recombinant nucleic acid and the recombinant nucleic acid targeting system provided herein comprise a polynucleotide encoding a guide RNA. In another embodiment, the recombinant nucleic acid and the recombinant nucleic acid targeting system provided herein comprise one or more, two or more, three or more, four or more, five or more, six or more, seven or more, eight or more, nine or more, or ten or more polynucleotides encoding one or more, two or more, three or more, four or more, five or more, six or more, seven or more, eight or more, nine or more, or ten or more guide RNAs. In some embodiments, the polynucleotide encoding a guide RNA provided herein is operably linked to a promoter. In certain other embodiments, the polynucleotide encoding a guide RNA provided herein is operably linked to a U6 snRNA promoter. In yet another embodiment, the polynucleotide encoding a guide RNA provided herein is operably linked to a J23119 promoter. In other embodiments, the polynucleotide encoding a guide RNA provided herein is operably linked to a U6 snRNA promoter as described in WO20150131101, incorporated by reference herein. In another embodiment, the guide RNA provided herein is an isolated RNA. In certain other embodiments, the guide RNA provided herein is encoded in a vector, a plasmid, or a bacterial vector. In preferred embodiments, the gRNA comprises a CRISPR/Cas system associated RNA (crRNA) sequence and a trans-activating CRISPR/Cas system RNA (tracrRNA) sequence. In certain other embodiments provided herein, a guide RNA provided herein comprises a crRNA. In other embodiments, a guide RNA provided herein comprises a tracrRNA. In yet another embodiment, a guide RNA provided herein comprises a single-chain guide RNA (sgRNA). In specific embodiments, a single-chain guide RNA provided herein comprises both a crRNA and a tracrRNA. In other embodiments, a guide RNA provided herein comprises a trans-activating CRISPR RNA (tracrRNA) sequence, or other sequences and transcripts from a CRISPR locus. In some embodiments, a guide RNA provided herein does not comprise tracrRNA.

In some embodiments, the gRNA is capable of complexing with the Cas protein, and directing sequence specific binding of the gRNA-Cas protein complex to a target nucleic acid sequence. In some embodiments, the gRNA is capable of complexing with the Cas protein to form a gRNA-Cas protein complex. In certain preferred embodiments, the gRNA directs the Cas protein (e.g., a Cas12k protein) as described herein to a particular target sequence of a target polynucleotide. Those skilled in the art will understand that, in some embodiments, the gRNA sequence is site-specific. That is, in some embodiments, the gRNA associates specifically with one or more target nucleic acid sequences (e.g., specific DNA or genomic DNA sequences) and not to non-target sequences (e.g., non-specific DNA or random sequences).

In some embodiments, the composition as described herein comprises a gRNA that associates with the Cas protein described herein (e.g., Cas12k) and directs the Cas protein to a target sequence (e.g., DNA) of a target polynucleotide. The gRNA may associate with a target sequence and alter functionality of the Cas protein and or the at least one CRISPR-associated transposase protein(s) (e.g., alters affinity of the Cas12k, e.g., by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more).

The gRNA described herein may target (e.g., associate with, be directed to, contact, or bind) one or more nucleotides of a target sequence. In some embodiments, the transposase activity of the CRISPR-associated transposases described herein is activated upon formation of the Cas protein/gRNA RNP complex.

In some embodiments, the gRNA comprises a spacer sequence. In some embodiments, the spacer sequence of the gRNA may be generally designed to have a length of between 16-25 nucleotides (e.g., 16, 17, 18, 19, 20, 21, 22, 23, 24, or 25 nucleotides) and be complementary to a specific nucleic acid sequence. In some embodiments, the spacer sequence of the gRNA may be generally designed to have a length of up to about 35 nucleotides (e.g., 26, 27, 28, 29, 30, 31, 32, 33, 34, or 35 nucleotides) and be complementary to a specific nucleic acid sequence. In some particular embodiments, the gRNA may be designed to be complementary to a specific DNA strand, e.g., of a genomic locus. In some embodiments, the spacer sequence is designed to be complementary to a specific DNA strand, e.g., a specific genomic locus.

In certain embodiments, the gRNA includes or comprises a direct repeat sequence linked to a sequence or spacer sequence. In some embodiments, the gRNA includes a direct repeat sequence and a spacer sequence or a direct repeat-spacer-direct repeat sequence. In certain embodiments, the gRNA includes a truncated direct repeat sequence and a spacer sequence, which is typical of processed or mature crRNA. In other embodiments, the Cas protein forms a complex with the gRNA, and the gRNA directs the complex to associate with site-specific target nucleic acid that is complementary to at least a portion of the gRNA sequence.

In some embodiments, the gRNA comprises a sequence, e.g., RNA sequence, has at least about 80%, at least about 90%, at least about 95%, at least about 96%, at least about 97%, at least about 98%, at least about 99% complementary to a target sequence. In other embodiments, the gRNA comprises a sequence at least about 80%, at least about 90%, at least about 95%, at least about 96%, at least about 97%, at least about 98%, at least about 99% complementary to a DNA sequence. In another embodiment, the gRNA comprises a sequence at least about 80%, at least about 90%, at least about 95%, at least about 96%, at least about 97%, at least about 98%, at least about 99% complementary to a genomic sequence. In yet other embodiments, the gRNA comprises a sequence complementary to or a sequence comprising at least about 80%, at least about 90%, at least about 95%, at least about 96%, at least about 97%, at least about 98%, at least about 99% complementarity to a sequence set forth in SEQ ID NO: 5. In some embodiments, the gRNA comprises a sequence as set forth in SEQ ID NO: 5.

In some embodiments, the CRISPR-Cas system described herein includes one or more (e.g., two, three, four, five, six, seven, eight, or more) gRNA sequences. In some embodiments, the gRNA has an architecture similar to, for example International Publication Nos. WO 2014/093622 and WO 2015/070083, the entire contents of each of which are incorporated herein by reference.

In some embodiments, the Cas protein and the gRNA as described herein form a complex (e.g., a ribonucleoprotein (RNP)). In some embodiments, the complex includes other components (e.g., at least one CRISPR-associated transposase protein(s)). In some embodiments, the complex is activated upon binding to a target sequence that has complementarity to a sequence in the gRNA. In some embodiments, the target polynucleotide is a double-stranded DNA (dsDNA). In some embodiments, the target polynucleotide is a single-stranded DNA (ssDNA). In other embodiments, the sequence-specificity requires a complete match of a sequence in the gRNA to the target sequence. In yet other embodiments, the sequence specificity requires a partial (contiguous or non-contiguous) match of a sequence in the gRNA to the target sequence. In some embodiments, the complex becomes activated upon binding to the target sequence.

In certain other embodiments, the Cas protein described herein (e.g., Cas12k protein) binds to a target sequence at a sequence defined by the region of complementarity between the gRNA and the target polynucleotide. In some embodiments, the protospacer-adjacent motif (PAM) sequence recognized by the Cas protein described herein is located directly upstream of the target sequence of the target polynucleotide (e.g., directly 5′ of the target sequence). In some embodiments, the PAM sequence recognized by the Cas protein described herein is located directly 5′ of the non-complementary strand (e.g., non-target strand) of the target polynucleotide. In certain embodiments described herein, the Cas protein targets a sequence adjacent to a PAM, wherein the PAM comprises the nucleotide sequence 5′-GGTT-3′. In certain embodiments, the PAM comprises the nucleotide sequence 5′-GTN-3′, 5′-NGTN-3′, or 5′-GGTN-3′. In certain embodiments, the PAM comprises the nucleotide sequence 5′-GGTT-3′. In certain embodiments, the PAM comprises the nucleotide sequences 5′-GTT-3′, 5′-GTA-3′, 5′-GTC-3′, or 5′-GTG-3′. In certain embodiments, the PAM comprises 5′-GGTA-3′, 5′-GGTC-3′, or 5′-GGTG-3′. As used herein, the “complementary strand” hybridizes to the RNA guide. As used herein, the “non-complementary strand” does not directly hybridize to the RNA.

In certain embodiments, the insertion of a target sequence into a target polypeptide occurs at the Cas binding site. In other embodiments, the insertion occurs at a position distal to a Cas binding site on a nucleic acid molecule. In some embodiments, the insertion may occur at a position on the 3′ side from a Cas binding site, e.g., at least about 1 base pair (bp), at least about 5 bp, at least about 10 bp, at least about 15 bp, at least about 20 bp, at least about 35 bp, at least about 40 bp, at least about 45 bp, at least about 50 bp, at least about 55 bp, at least about 60 bp, at least about 65 bp, at least about 70 bp, at least about 75 bp, at least about 80 bp, at least about 85 bp, at least about 90 bp, at least about 95 bp, or at least about 100 bp on the 3′ side from a Cas binding site.

In some embodiments, binding of the Cas protein/gRNA blocks access of one or more endogenous cellular molecules or pathways to the target sequence, thereby modifying the target sequence. For example, binding of a the Cas protein/gRNA may block endogenous transcription or translation machinery thereby decreasing the expression of the target nucleic acid. Nucleic acid molecules encoding the Cas protein described herein can further be codon-optimized. The nucleic acid can be codon-optimized for use in a particular host cell, such as a bacterial cell.

In some embodiments, the present disclosure provides a recombinant nucleic acid targeting system comprising at least one of the CRISPR-associated transposase proteins (e.g. TniA, TniB, and TniQ), a Cas12k, and a guide RNA (gRNA). In other embodiments, the present disclosure provides a recombinant nucleic acid targeting system comprising at least two of the CRISPR-associated transposase proteins (e.g., TniA, TniB, and TniQ), and Cas12k, and guide RNA (gRNA). In certain other embodiments, the present disclosure provides a recombinant nucleic acid targeting system comprising TniA, TniB, TniQ, a Cas12k, and a guide RNA (gRNA). The present disclosure also provides a recombinant nucleic acid targeting system for sequence-specific modification of a target sequence. In some embodiments, the biochemical characteristics of a CRISPR-associated transposase system of the present disclosure are analyzed in bacterial cells, as described in Example 1.

### C. Recombinant Nucleic Acid Compositions and Recombinant Nucleic Acid Targeting Systems

The recombinant nucleic acid compositions and recombinant nucleic acid targeting systems described herein comprise a CRISPR associated (Cas) protein (e.g., Cas12k protein), or a polynucleotide encoding a Cas protein and at least one CRISPR associated transposase protein, or a polynucleotide encoding at least one CRISPR associated transposase protein. For example, in some embodiments, the recombinant nucleic acid compositions and the recombinant nucleic acid targeting systems described herein comprise a Cas protein, a TniA, a TniB, and a TniQ. In certain embodiments, the recombinant nucleic acid compositions and the recombinant nucleic acid targeting systems described herein comprise a Cas protein, a TniA, a TniB, and a TniQ, wherein one of the protein sequences for the Cas protein, the TniA protein, the TniB protein, and the TniQ protein comprises an amino acid sequence that is at least 95% identical to an amino acid sequence set forth in SEQ ID NOs: 1, 2, 3, and 4, respectively, for the Cas protein, TniA protein, TniB protein, and TniQ protein.

In certain other embodiments, the recombinant nucleic acid targeting systems described herein comprise one or more of a Cas protein (e.g., Cas12k protein), a TniA, TniB, and a TniQ, and further comprise at least one nucleic acid sequence encoding a transposon left end (TE-L) and a nucleic acid sequence encoding a transposon right end (TE-R). In some embodiments, the recombinant nucleic acid targeting systems described herein comprise a TniA and a TE-L and a TE-R. In some embodiments, the preferred TE-L and TE-R is determined by the TniA of the recombinant nucleic acid targeting system. For example, in some embodiments, the recombinant nucleic acid targeting system comprises a TniA as described in SEQ ID NO: 2 (i.e., a TniA comprising an amino acid sequence having at least about 80% sequence identity, at least about 85% sequence identity, at least about 90% sequence identity, at least about 95% sequence identity, at least about 99% sequence identity, or about 100% sequence identity to SEQ ID NO: 2), a TE-L (i.e., a TE-L comprising a nucleotide sequence having at least about 80% sequence identity, at least about 85% sequence identity, at least about 90% sequence identity, at least about 95% sequence identity, at least about 99% sequence identity, or about 100% sequence identity to SEQ ID NO: 6) and a TE-R (i.e., a TE-R comprising a nucleotide sequence having at least about 80% sequence identity, at least about 85% sequence identity, at least about 90% sequence identity, at least about 95% sequence identity, at least about 99% sequence identity, or about 100% sequence identity to SEQ ID NO: 7). In certain embodiments, the recombinant nucleic acid targeting systems described herein comprise a TniA and a donor polynucleotide, wherein the donor polynucleotide comprises a payload sequence for insertion into the target sequence, a TE-L nucleic acid sequence that is at least 95% identical to a nucleic acid sequence set forth in SEQ ID NO: 6, and a TE-R nucleic acid sequence that is at least 95% identical to a nucleic acid sequence set forth in SEQ ID NO: 7.

### D. Target Polynucleotides

The recombinant nucleic acid targeting systems described herein may further comprise a target polynucleotide comprising a target sequence capable of hybridizing to a gRNA. A target polynucleotide may be an equivalent of a target site into which a transposable element is inserted. In certain embodiments of the recombinant nucleic acid targeting system described herein, the target polynucleotide comprises a protospacer-adjacent motif (PAM) sequence and a target sequence capable of hybridizing to a gRNA. As described herein, a “target sequence” refers to a sequence to which the gRNA sequence has (or is designed to have) complementarity. The hybridization between a target sequence and its complementary sequence in a gRNA facilitates the formation of a Cas/gRNA/target sequence complex. In other embodiments, the target polynucleotide provided herein is operably linked to a promoter. In yet other embodiments, the target polynucleotide described herein comprises at least a PAM sequence with a nucleotide sequence comprising 5′-GGTT-3′. In certain embodiments, the PAM comprises the nucleotide sequence 5′-GTN-3′, 5′-NGTN-3′, or 5′-GGTN-3′. In certain embodiments, the PAM comprises the nucleotide sequence 5′-GGTT-3′. In certain embodiments, the PAM comprises the nucleotide sequences 5′-GTT-3′, 5′-GTA-3′, 5′-GTC-3′, or 5′-GTG-3′. In certain embodiments, the PAM comprises 5′-GGTA-3′, 5′-GGTC-3′, or 5′-GGTG-3′. In some embodiments, the PAM may be a 5′ PAM sequence (i.e., located upstream of the 5′ end of the protospacer). The target polynucleotide sequence may comprise single- or double-stranded DNA. In some embodiments, formation of a complex comprising a CRISPR-associated (Cas) protein, gRNA, and CRISPR-associated transposase protein(s) results in insertion of a donor polynucleotide in one or both strands in or near (e.g. within about 1, about 2, about 3, about 4, about 5, about 6, about 7, about 8, about 9, about 10, about 20, about 50, 55, 60, 65, 70, 75, 80 or more base pairs from) a target sequence of a target polynucleotide. In other embodiments, formation of a complex comprising the Cas protein/gRNA RNP complex and at least one CRISPR-associated transposase protein(s) results in insertion of the donor polynucleotide in one or both strands in or near (e.g., within 1-10 base pairs, 5-15 base pairs, 10-20 base pairs, 15-25 base pairs, 20-30 base pairs, 25-35 base pairs, 30-40 base pairs, 35-45 base pairs, 45-60 base pairs, 45-70 base pairs, 45-80 base pairs or more base pairs from) a target sequence of a target polynucleotide.

### E. Donor Polynucleotides

The recombinant nucleic acid targeting systems described herein may further comprise a donor polynucleotide comprising a payload sequence for insertion into a target polynucleotide. A donor polynucleotide may be an equivalent of a transposable element that is capable of being integrated into a target sequence. A donor polynucleotide may be any type of polynucleotide that includes a payload sequence, e.g., a gene, a gene fragment, a non-coding polynucleotide, a regulatory polynucleotide, a synthetic polynucleotide, and fragments or components thereof. More specifically, the term “donor polynucleotide”, as described herein, refers to a polynucleotide molecule that includes a payload sequence capable of being inserted into a target nucleic acid using a CRISPR-associated transposase, or a method, as described herein. In some embodiments, the payload sequence provided herein is operably linked to a promoter. In some embodiments, the donor polynucleotide comprises a nucleic acid sequence encoding a transposon left end (TE-L) and a nucleic acid sequence encoding a transposon right end (TE-R). The term “transposon end sequences”, as used herein, refers to nucleotide sequences that are necessary to form a complex with the CRISPR-associated transposase protein(s) that is functional as determined using an in vitro or in vivo transposition reaction. The TE-R and TE-L sequences typically flank a payload sequence of a donor polypeptide as inverted repeats, a feature recognized by the CRISPR-associated transposase protein, which facilitates insertion of the payload sequence into the target sequence of the target polynucleotide. In some embodiments, the TE-L comprises a nucleic acid set forth in SEQ ID NO: 6 and the TE-R comprises a nucleic acid set forth in SEQ ID NO: 7.

In certain other embodiments, the payload sequence of the donor polynucleotide is inserted into the target polynucleotide via a co-integration mechanism. For example, the donor polynucleotide and the target polynucleotide may be nicked and fused. A duplicate of the fused donor polynucleotide and the target polynucleotide may be generated by a polymerase. In other embodiments, the donor polynucleotide is inserted in the target polynucleotide via a cut and paste mechanism. For example, the donor polynucleotide may be comprised in a nucleic acid molecule and may be cut out and inserted to another position in the nucleic acid molecule.

### F. Vectors

The present disclosure provides one or more vectors comprising the recombinant nucleic acid and/or the recombinant nucleic acid targeting system described herein. In some embodiments, the disclosure provides one or more vectors for expressing the recombinant nucleic acid or the recombinant nucleic acid targeting system described herein. The vectors provided herein are also used in the methods for modifying a target polynucleotide as described herein. In one embodiment, a vector provided herein includes a first promoter operably linked to a first polynucleotide encoding at least one CRISPR-associated transposase protein or functional fragment thereof, and a Cas protein (e.g., Cas12k protein). In the embodiment described above, the vector also includes a second promoter operably linked to a second polynucleotide encoding a guide RNA (gRNA). Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. In some embodiments, the vectors described herein are plasmids. The term “plasmid”, as used herein, refers to a circular double stranded DNA loop into which additional DNA segments can be inserted using, for example, standard molecular cloning techniques. In certain embodiments described herein, the vectors are “expression vectors” capable of directing the expression of genes to which they are operatively-linked. Typical expression vectors, including certain vectors described herein, include transcription and translation terminators, initiation sequences, and promoters that are useful for expression of the desired polynucleotide. Expression of natural or synthetic polynucleotides is typically achieved by operably linking a polynucleotide encoding the natural or synthetic polynucleotides to a promoter and incorporating the construct into an expression vector. In one particular embodiment, expression of one or more genes of interest, e.g., one or more polynucleotide(s) encoding TniA, TniB, TniQ, Cas12k, is typically achieved by operably linking one or more polynucleotide(s) encoding the one or more genes of interest, e.g., one or more polynucleotide(s) encoding TniA, TniB, TniQ, Cas12k to a promoter and incorporating the construct into an expression vector (see, e.g. pEffector plasmid A2 as described herein).

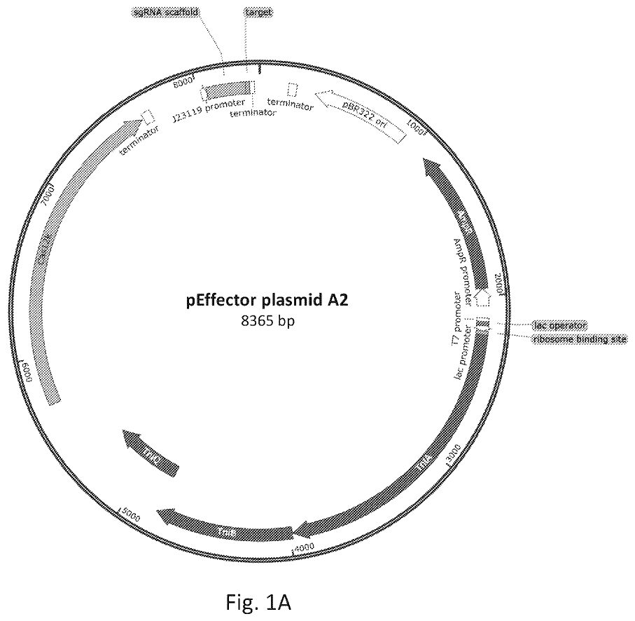

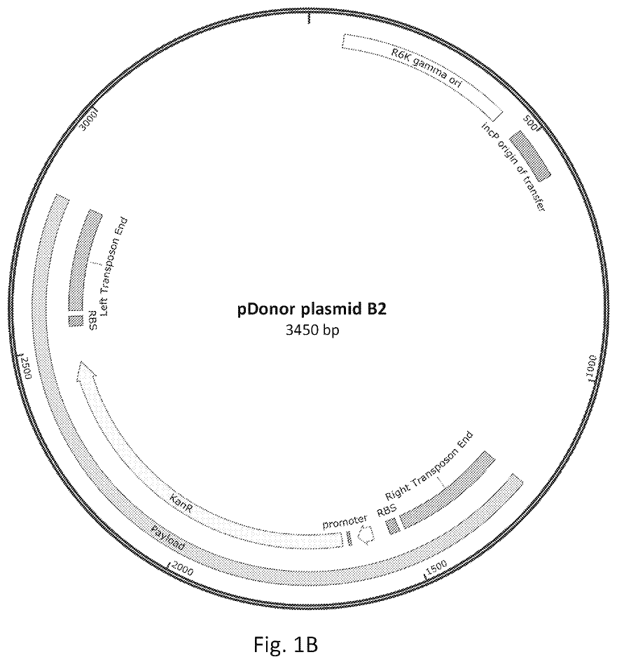

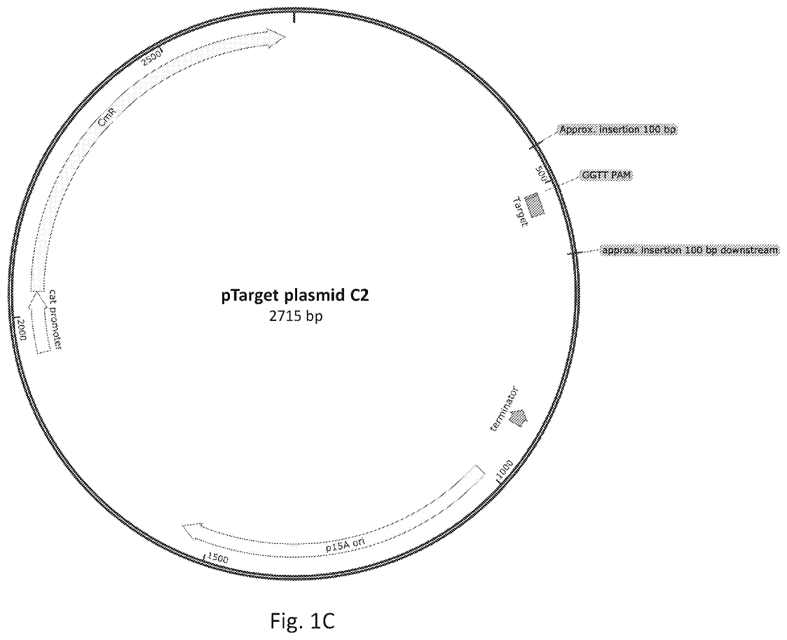

In particular embodiments, one or more of the components of the compositions and systems described herein were expressed on expression plasmids. In one particular embodiment, the disclosure provides a pEffector plasmid A2 as shown in FIG. 1A. In another embodiment, the pEffector plasmid A2 comprises polynucleotides encoding the amino acid sequences of a Cas12k protein, a TniA protein, a TniB protein, and a TniQ protein. In yet another embodiment, the pEffector plasmid A2 comprises polynucleotides encoding the amino acid sequences of a Cas12k protein (SEQ ID NO: 1), a TniA protein (SEQ ID NO: 2), a TniB protein (SEQ ID NO: 3), and a TniQ protein (SEQ ID NO: 4) as shown in Table 1 and an ampicillin resistance protein (AmpR).

In other embodiments, the pEffector plasmid further comprises a polynucleotide encoding a gRNA. In one embodiment, the gRNA comprises a polynucleotide encoding a crRNA. In another embodiment, the gRNA comprises a polynucleotide encoding a tracrRNA. In yet another embodiment, the gRNA comprises a single-guide RNA (sgRNA) sequence comprising a polynucleotide encoding a crRNA, a polynucleotide encoding a tracrRNA and a spacer sequence. In particular embodiments, the sgRNA sequence comprises a nucleotide sequence as set forth in SEQ ID NO: 5 shown in Table 1. The spacer sequence in SEQ ID NO: 5 is represented as N's.

In other embodiments, the disclosure provides a pDonor plasmid comprising a payload sequence. In one particular embodiment, the disclosure provides a pDonor plasmid B2 as shown in FIG. 1B comprising coding regions for a payload sequence and a kanamycin resistance protein, and further comprising the sequences of left (TE-L) and right (TE-R) transposon ends. In particular embodiments, the TE-L comprises a nucleic acid sequence as set forth in SEQ ID NO: 6 (Table 1). In particular embodiments, the TE-R comprises a nucleic acid sequence as set forth in SEQ ID NO: 7 (Table 1).

In other embodiments, the disclosure provides a pTarget plasmid comprising a target sequence. In one particular embodiment, the disclosure provides a pTarget plasmid C2 as shown in FIG. 1C comprising a target sequence and a protospacer-adjacent motif (PAM) sequence. In another embodiment, the PAM sequence comprises the nucleotide sequence 5′-GGTT-3′. In certain embodiments, the PAM comprises the nucleotide sequence 5′-GTN-3′, 5′-NGTN-3′, or 5′-GGTN-3′. In certain embodiments, the PAM comprises the nucleotide sequence 5′-GGTT-3′. In certain embodiments, the PAM comprises the nucleotide sequences 5′-GTT-3′, 5′-GTA-3′, 5′-GTC-3′, or 5′-GTG-3′. In certain embodiments, the PAM comprises 5′-GGTA-3′, 5′-GGTC-3′, or 5′-GGTG-3′.

In some embodiments, the present disclosure provides a cell comprising recombinant nucleic acids and/or the recombinant nucleic acid targeting systems described herein. In some embodiments, the cell is a prokaryotic cell. In certain embodiments, the cell is a bacterial cell or a cell that is derived from a bacterial cell. In other embodiments, the one or more nucleic acids, plasmids, and/or vectors for expressing the recombinant nucleic acids and/or the recombinant nucleic acid targeting systems described herein are introduced into a bacterial cell. In another embodiment, the nucleic acids, plasmids, and/or vectors provided herein are transformed into a bacterial cell. The nucleic acids, plasmids, and/or vectors that are typically suited for expression in bacterial cells can be appropriately selected. Techniques for introducing the one or more nucleic acids, plasmids, and/or vectors described herein include, but are not limited to, heat-shock and electroporation, and are techniques well known to a person of skill in the art. In some embodiments, the bacterial cell is an E. coli cell. In some embodiments, the E. coli cell is a pir-116D strain (e.g., PIR1). In one embodiment, the pEffector plasmid A2 is introduced into a bacterial cell. In another embodiment, the pDonor plasmid B2 is introduced into a bacterial cell. In yet another embodiment, the pTarget plasmid C2 is introduced into a bacterial cell. In a preferred embodiment, the pEffector plasmid A2, the pDonor plasmid B2 and the pTarget plasmid C2 are introduced into the same bacterial cell. In another embodiment, the pEffector plasmid A2, the pDonor plasmid B2 and the pTarget plasmid C2 are introduced into the same bacterial cell simultaneously. In another embodiment, the pEffector plasmid A2, the pDonor plasmid B2 and the pTarget plasmid C2 are introduced into the same bacterial cell sequentially.

In some embodiments, the nucleic acids, plasmids, and/or vectors provided herein further comprise a selectable marker gene and/or a reporter gene to facilitate identification and selection of cells comprising the nucleic acids, plasmids, and/or vectors. Both selectable markers and reporter genes may be flanked with appropriate transcriptional control sequences to enable expression in cell. Examples of a suitable selectable marker includes a nucleic acid sequence encoding an appropriate antibiotic resistance protein, e.g., an ampicillin resistance protein, a kanamycin resistance protein, and the like. By use of such a selection marker, successful incorporation of the nucleic acids, plasmids, and/or vectors comprising recombinant nucleic acids and/or the recombinant nucleic acid targeting systems described herein can be confirmed by survival of cells in the presence of the antibiotic. Examples of a suitable reporter gene includes a nucleic acid sequence encoding a fluorescent protein, e.g. green fluorescent protein (GFP), and the like. By use of such a reporter gene, successful incorporation of the nucleic acids, plasmids, and/or vectors described herein can be confirmed by observation of the expression of the fluorescent protein.

### G. Method for Modifying a Target Polynucleotide

The present disclosure further provides methods for modifying a target polynucleotide in a bacterial cell, which comprises introducing into a bacterial cell, a first recombinant nucleic acid comprising at least one CRISPR-associated transposase protein or a polynucleotide encoding the at least one CRISPR-associated transposase protein, a Cas protein (e.g., Cas12k protein) or a polynucleotide encoding the Cas protein and a guide RNA (gRNA) or a polynucleotide encoding the gRNA; a second recombinant nucleic acid comprising a target polynucleotide; and a third recombinant nucleic acid comprising a donor polynucleotide.

The recombinant nucleic acids described herein may be introduced into a bacterial cell or population of bacterial cells by transforming one or more delivery polynucleotides (e.g., plasmids) comprising nucleic acid sequences encoding the recombinant nucleic acids described herein. The nucleic acid sequences encoding the recombinant nucleic acids described herein may be expressed from their nucleic acid sequences when operably linked to one or more regulatory sequences (e.g., promoters) that control the expression of proteins and nucleic acids in the bacterial cell or population of bacterial cells. The recombinant nucleic acids described herein may be encoded on the same delivery polynucleotide, on individual delivery polynucleotides, or a combination thereof. In some embodiments, the delivery polynucleotides may be a vector. In other embodiments, the delivery polynucleotides are plasmids. In yet other embodiments, the delivery polynucleotides are plasmids or are a combination of vectors and plasmids. Exemplary vectors and plasmids are provided are described herein.

In certain embodiments, the disclosure provides a method for modifying a target polynucleotide in a bacterial cell comprising introducing a recombinant nucleic acid encoding the at least one CRISPR-associated transposase protein, wherein a recombinant nucleic acid encoding the at least one CRISPR-associated transposase protein is operatively linked to at least one heterologous promoter (e.g., a T7 promoter). In some embodiments, the at least one CRISPR-associated transposase protein is provided by expressing in the bacterial cell a recombinant DNA molecule encoding the at least one CRISPR-associated transposase protein operatively linked to at least one heterologous promoter (e.g., a T7 promoter). In other embodiments, the at least one CRISPR-associated transposase protein is provided by transforming into the bacterial cell a plasmid comprising a DNA molecule encoding the at least one CRISPR-associated transposase protein operatively linked to at least one heterologous promoter (e.g., a T7 promoter). In certain other embodiments, the at least one CRISPR-associated transposase protein is provided by introducing into the bacterial cell a composition comprising a RNA molecule encoding the at least one CRISPR-associated transposase protein.

In some embodiments, the methods for modifying a target polynucleotide in a bacterial cell provided herein comprise introducing into the bacterial cell a recombinant nucleic acid encoding at least one CRISPR-associated transposase protein selected from the group consisting of a TniA protein, a TniB protein, and a TniQ protein. In other embodiments, the methods provided herein comprise introducing into the bacterial cell a polynucleotide encoding at least two CRISPR-associated transposase proteins selected from the group consisting of a TniA protein, a TniB protein, and a TniQ protein. In yet another embodiment, the methods provided herein comprise introducing into the bacterial cell a polynucleotide encoding three CRISPR-associated transposase proteins selected from the group consisting of a TniA protein, a TniB protein, and a TniQ protein. In some embodiments, the methods provided herein comprise introducing into the bacterial cell a polynucleotide encoding a CRISPR-associated transposase protein comprising an amino acid sequence having at least about 50%, about 55%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, or at least about 99.5% or more amino acid sequence identity to a TniA protein comprising an amino acid sequence as set forth in SEQ ID NO: 2. In other embodiments, the methods provided herein comprise introducing into the bacterial cell a polynucleotide encoding a CRISPR-associated transposase protein comprising an amino acid sequence that is about 100% identical to a TniA protein comprising the amino acid sequence as set forth in SEQ ID NO: 2. In certain other embodiments, the methods provided herein comprise introducing into the bacterial cell a polynucleotide encoding a CRISPR-associated transposase protein comprising an amino acid sequence having at least about 50%, about 55%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or at least about 99.5% or more amino acid sequence identity to a TniB protein comprising an amino acid sequence as set forth in SEQ ID NO: 3. In another embodiment, the methods provided herein comprise introducing into the bacterial cell a polynucleotide encoding a CRISPR-associated transposase protein comprising an amino acid sequence having that is about 100% identical to a TniB protein comprising an amino acid sequence as set forth in SEQ ID NO: 3. In certain other embodiments, the methods provided herein comprise introducing into the bacterial cell a polynucleotide encoding a CRISPR-associated transposase protein comprising an amino acid sequence having at least about 50%, about 55%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or at least about 99.5% or more amino acid sequence identity to a TniQ protein comprising an amino acid sequence as set forth in SEQ ID NO: 4. In other embodiments, the methods provided herein comprise introducing into the bacterial cell a polynucleotide encoding a CRISPR-associated transposase protein comprising an amino acid sequence that is about 100% identical to a TniQ protein comprising an amino acid sequence as set forth in SEQ ID NO: 4.

In certain embodiments, the disclosure provides a method for modifying a target polynucleotide in a bacterial cell further comprising introducing into the bacterial cell a recombinant nucleic acid encoding at least one CRISPR-associated transposase protein and a Cas protein (e.g., Cas12k), wherein a recombinant nucleic acid encoding the at least one CRISPR-associated transposase protein and the Cas protein is operatively linked to at least one heterologous promoter (e.g., a T7 promoter). In some embodiments, the at least one CRISPR-associated transposase and the Cas protein are provided by expressing in the bacterial cell a recombinant DNA molecule encoding the at least one CRISPR-associated transposase and a recombinant DNA molecule encoding the Cas protein, each operatively linked independently to at least one heterologous promoter. In some embodiments, the methods provided herein comprise introducing into the bacterial cell a recombinant nucleic acid encoding the Cas protein comprising an amino acid sequence comprising at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% or more sequence identity to the amino acid sequence of a Cas12k protein as set forth in SEQ ID NO: 1. In certain other embodiments, the methods provided herein comprise introducing into the bacterial cell a recombinant nucleic acid encoding the Cas protein comprising an amino acid sequence that is about 100% sequence identity to the amino acid sequence of a Cas12k protein comprising an amino acid sequence as set forth in SEQ ID NO: 1.

In certain embodiments, the disclosure provides a method for modifying a target polynucleotide in a bacterial cell comprising introducing into the bacterial cell a recombinant nucleic acid encoding at least one CRISPR-associated transposase protein, a Cas protein (e.g., Cas12k), and a guide RNA (gRNA), wherein a recombinant nucleic acid encoding the at least one CRISPR-associated transposase protein and the Cas protein is operatively linked to a heterologous promoter (e.g., a T7 promoter) and wherein the recombinant nucleic acid encoding the gRNA is operably linked to a different heterologous promoter (e.g., a J23119 promoter). In some embodiments, the disclosure provides a method for introducing into the bacterial cell a recombinant nucleic acid encoding the at least one CRISPR-associated transposase protein, the Cas protein (e.g., Cas12k), and the guide RNA (gRNA) on a more than one plasmid. In certain preferred embodiments, the disclosure provides a method for introducing into the bacterial cell a recombinant nucleic acid comprising encoding the at least one CRISPR-associated transposase protein, the Cas protein (e.g., Cas12k), and the guide RNA (gRNA) on a single plasmid. In a particular embodiment, the at least one CRISPR-associated transposase protein, the Cas protein (e.g., Cas12k), and the guide RNA (gRNA) are encoded on a single plasmid (pEffector plasmid A2) as shown in FIG. 1A. In other embodiments, the at least one CRISPR-associated transposase protein, the Cas protein (e.g., Cas12k), and the guide RNA (gRNA) are introduced into a bacterial cell as a pre-formed ribonucleoprotein (RNP) complex. In yet another embodiment, the Cas protein and the guide RNA (gRNA) are introduced into a bacterial cell as a pre-formed ribonucleoprotein (RNP) complex and the at least one CRISPR-associated transposase protein is introduced into the bacterial cell as a recombinant nucleic acid encoding the at least one CRISPR-associated transposase protein.

In some embodiments, the methods provided herein comprise introducing into a bacterial cell a recombinant nucleic acid encoding a gRNA a sequence, wherein the gRNA sequence is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5% or more complementary to a target sequence of a target polynucleotide. In some embodiments, the gRNA comprises a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at about least 99%, at least about 99.5% or more complementary to a DNA sequence. In certain other embodiments, the gRNA comprises a sequence that is at least about 80%, at least about 81%, at least about 82%, at least about 83%, at least about 84%, at least about 85%, at least about 86%, at least about 87%, at least about 88%, at least about 89%, at least 90%, at least about 91%, at least about 92%, at least about 93%, at least about 94%, at least about 95%, at least about 96%, at least about 97%, at least about 98%, at least about 99%, at least about 99.5% at least about 99.5% or more or more complementary to a genomic sequence. In some embodiments, the gRNA comprises a sequence complementary to or a sequence comprising at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5% or more complementarity to a sequence set forth in SEQ ID NO: 5. In some embodiments of the methods described herein, the gRNA comprises a sequence as set forth in SEQ ID NO: 5.

In certain embodiments, the method further comprises introducing into a bacterial cell a recombinant nucleic acid comprising a target polynucleotide, wherein the target polynucleotide comprises a target sequence capable of hybridizing to the gRNA, and comprises a protospacer-adjacent motif (PAM) sequence. In certain embodiments, target sequence is operably linked to a heterologous promoter (e.g., a cat promoter). In other embodiments, the PAM sequence is a nucleotide sequence comprising 5′-GGTT-3′. In certain embodiments, the PAM comprises the nucleotide sequence 5′-GTN-3′, 5′-NGTN-3′, or 5′-GGTN-3′. In certain embodiments, the PAM comprises the nucleotide sequence 5′-GGTT-3′. In certain embodiments, the PAM comprises the nucleotide sequences 5′-GTT-3′, 5′-GTA-3′, 5′-GTC-3′, or 5′-GTG-3′. In certain embodiments, the PAM comprises 5′-GGTA-3′, 5′-GGTC-3′, or 5′-GGTG-3′. In another embodiment, the disclosure provides a method for modifying a target polynucleotide in a bacterial cell comprising introducing into the bacterial cell a target polypeptide using a single plasmid. In a particular embodiment, the single plasmid is a pTarget plasmid C2 as shown in FIG. 1C.

In certain embodiments, the method further comprises introducing into a bacterial cell a recombinant nucleic acid comprising a donor polynucleotide. In preferred embodiment, the donor polynucleotide comprises a payload sequence for insertion into the target sequence of a target polynucleotide. In another embodiment, the payload sequence is operably linked to a heterologous promoter. In some embodiments, the donor polynucleotide further comprises a nucleic acid sequence encoding a transposon left end (TE-L) and a nucleic acid sequence encoding a transposon right end (TE-R). In specific embodiments, the TE-L and TE-R sequences are at least about 50%, about 55%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 99.5% or more identical to the nucleic acid sequences of a TE-L and a TE-R as set forth in SEQ ID NO: 6 and SEQ ID NO: 7, respectively. In some embodiments, the TE-L has a nucleic acid as set forth in SEQ ID NO: 6 and the TE-R has a nucleic acid set as set forth in SEQ ID NO: 7. In certain embodiments, the disclosure provides a method for modifying a target polynucleotide in a bacterial cell comprising introducing into the bacterial cell a donor polypeptide using a single plasmid. In a particular embodiment, the single plasmid is a pDonor plasmid B2 as shown in FIG. 1B.

In some embodiments, the method described herein comprises modifying a target polynucleotide by introducing into a bacterial cell, a first recombinant nucleic acid comprising (i) a polynucleotide encoding at least one CRISPR-associated transposase protein, (ii) a polynucleotide encoding a CRISPR associated (Cas) protein, and (iii) a polynucleotide encoding a guide RNA (gRNA); a second recombinant nucleic acid comprising a target polynucleotide; and a third recombinant nucleic acid comprising a donor polynucleotide, as described herein. In some embodiments, the first recombinant nucleic acid, the second recombinant nucleic acid and the third recombinant nucleic acid are simultaneously introduced into the bacterial cell. In certain other embodiments, the first recombinant nucleic acid, the second recombinant nucleic acid and the third recombinant nucleic acid are sequentially introduced into the bacterial cell. In yet another embodiment, the methods described herein comprise modifying a target polynucleotide by independently introducing into the bacterial cell, each of the first recombinant nucleic acid, the second recombinant nucleic acid and the third recombinant nucleic acid described above. In certain other embodiments, the method described herein comprises modifying a target polynucleotide by introducing into a bacterial cell, a pEffector plasmid A2 as shown in FIG. 1A, a pDonor plasmid B2 shown in FIG. 1B and a pTarget plasmid C2 as shown in FIG. 1C. In preferred embodiments, the bacterial cell is an E. coli cell. In other embodiments, the E. coli cell is a cell from a pir-116D strain (e.g. PIR1). In other embodiments, the pEffector plasmid A2, the pDonor plasmid B2 and the pTarget plasmid C2, are introduced into the same bacterial cell simultaneously. In other embodiments, the pEffector plasmid A2, the pDonor plasmid B2 and the pTarget plasmid C2, are introduced into the same bacterial cell sequentially. The methods disclosed herein further provide for the identification of the modification introduced into the target polynucleotide and the determination of % integration to the payload sequence into the target polynucleotide using sequencing analysis (e.g., nextseq NGS sequencing) and/or bioinformatics analysis (e.g., multiple sequence alignments) known to a person of skill in the art.

In some embodiments, the methods described herein include methods that comprise modifying a target polynucleotide by allowing at least one CRISPR-associated transposase protein, a Cas protein (e.g., Cas12k protein) and a gRNA as described herein to bind to a target sequence to facilitate insertion of a donor polypeptide into said target sequence, thereby modifying the target sequence. In another embodiment, the disclosure further provides a method of repairing a genetic locus in a bacterial cell using the recombinant nucleic acid targeting system described herein. In another embodiment, the disclosure provides methods of modifying a target polynucleotide (e.g., DNA) in a bacterial cell, wherein the method is an in vivo method, an ex vivo method or an in vitro method.

All references and publications cited herein are hereby incorporated by reference.

### EXAMPLES

The following examples are provided to further illustrate certain embodiments of the present disclosure, but are not intended to limit the scope of the disclosure. It will be understood by their exemplary nature that other procedures, methodologies, or techniques known to those skilled in the art may alternatively be used.

### Example 1—Determination of Transposase Activity in E. coli

This Example describes introduction of the CRISPR-associated trans system into E. coli to test transposase activity.

Each of the four proteins, Cas12k, TniA, TniB, and TniQ, were cloned into a plasmid referred to herein as “pEffector plasmid A2.” The schematic of pEffector plasmid A2 is shown in FIG. 1A, and the amino acid sequences of the Cas12k, TniA, TniB, and TniQ proteins are shown in Table 1. pEffector plasmid A2 further comprises a single-guide RNA (sgRNA) sequence containing the targeting sequence (e.g., the spacer). In the sgRNA sequence of SEQ ID NO: 5, the spacer sequence is represented as N's.

TABLE 1
Components of pEffector plasmid A2

| Protein | Sequence |
|---|---|
| Cas12k (SEQ ID NO: 1) | MSQITIQCCLIASESTRQKL |
| | WKLMAHLNTPLINELLQQLS |
| | KHPDFEKWRKNGKLPSTVVN |
| | QLCQPLKTDPSFTGQPSRLY |
| | LSAIHVVDYIYKSWLAIQKR |
| | LQQQLDGKIRWLEMLNSDAE |
| | LIEISGCSLEAIRTKAAEIL |
| | AIATPDSDVAAPLTKTGKAK |
| | KSKKSSASNPDRSLSHKLFD |
| | AYQETDDILSRSAISYLLRN |
| | GCKLNDKEEDLEKFAKRRRK |
| | VEIQIQRLTDKLTSRIPKGR |
| | DLTNAKWLETLFTATTTVPE |
| | DNVEAKRWQDILLTRSSSVP |
| | FPLIFETNEDLVWSKNEKGR |
| | LCVHFNGLSDLTFEVYCDRR |
| | QLHWFKRFLEDQQTKRKSKN |
| | QHSSGLFTLRNGRLAWQEGE |
| | GKGEPWQINRLTLYCCVDNR |
| | LWSAEGTEQVRQEKEEEITK |
| | FITKMNEKSDLSETQQAFIK |
| | RKESTLTRINNSFERPSQFL |
| | YQGQSHILVGVSLGLEKPAT |
| | VAVVDAIAGKVLAYRSIKQL |
| | LGDNYELLNRQRRQQQYLSH |
| | ERHKAQKSFSPNQFGTSELG |
| | QYVDRLLAKEIIAIAQTHKA |
| | GSIVLPKLGDMREIVQSEIQ |
| | AIAEEKFPGYVEGQQKYAKQ |
| | YRVNVHGWSHSRLIQSIQSK |
| | AAQIGIVIEEGKQPIRGSPQ |
| | DKAKELALSAYNLRLARRS |
| TniA (SEQ ID NO: 2) | MNSQQNPDLAVHTSAIP |
| | TEGLQEESDKPLERNVIVTA |
| | LSEEAQLKLEVIQSLLEKSD |
| | RTTYGQKLKEAAEKLSVSVR |
| | TVQRLVKKWEQDGVVGFTQT |
| | GRADKGRHRIGEFWENFILK |
| | TYREGNKGSKRMTPKQVALR |
| | VQAKARELEDSKPPNYRTVL |
| | RVLAPVLEKQEKAKSIRSPG |
| | WRGTTLSVKTREGKDLSVDY |
| | SNHVWQCDHTRVDVLLVDQH |
| | GQLLSRPWLTTVIDTYSRCI |
| | MGINLGFDAPSSVVVALALR |
| | HAILPKQYGSEYKLHCEWGT |
| | YGKPEHFYTDGGKDFRSNHL |
| | SQIGAQLGFVCHLRDRPSEG |
| | GVVERPFKTLNDQLFSTLPG |
| | YTGSNVQERPEDAEKDARLT |
| | LRELEQLLVRYLVDRYNQSI |
| | DARMGDQTRFERWEAGLPTV |
| | PVPIPERDLDICLMKQSRRT |
| | VQRGGCLQFQNLMYQGEYLA |
| | GYAGETVNLRFDPRDITTIL |
| | VYRQENNQEVFLTRAHAQGL |
| | ETEQLALDEAEAASRRLRTA |
| | GKTISNQSLLQEVVDRDALV |
| | ATKKSRKERQKLEQAVLRSA |
| | GVDESKTESLSSQVVEPDEV |
| | ESTAEINSQYEDMEVWDYEQ |
| | LREEYGF |
| TniB (SEQ ID NO: 3) | MTEASAIAKQLGGVKPDDEW |
| | LQAEIARLKGKSIVPLQQVK |
| | TLHDWLDGKRKARQSCRVVG |
| | ESRTGKTVACDAYRYRHKPH |
| | QEVGRPPIVPVVYIQPPQKC |
| | GSKDLFKEMIEYLKFRATKG |
| | TVSDFRGRAMEVLKGCEVEM |
| | LIIDEADRLKPETFAEVRDI |
| | YDKLGIAVVLVGTDRLEAVI |
| | KRDEQVYNRFRACHRFGKLS |
| | GKDFQDTVQAWEDRVLKLPV |
| | SSNLTSKDMLRILTLATEGY |
| | IGRLDEILRETAIRSLSKGF |
| | KKIDKAVLQEVAKEYK |
| TniQ (SEQ ID NO: 4) | MTAPDVKPWLFIIEPYPGES |
| | LSHFLGRFRRANHLSAAGLG |
| | NLAGIGAVVARWERFHFNPR |
| | PSQQELEAIASVVEVDADRL |
| | AQMLPPLGVGMQHEPIRLCG |
| | ACYAVTPCHQIEWQFKEMGG |
| | CNRHNLRLLSKCPKCEDRFK |
| | IPALWEYGLCGRCRTPFAEM |
| | AECQKRI |
| sgRNA (SEQ ID NO: 5) | AUAAUAAAUAGCGCCGCAGU |
| | UCAUGCUGCUUGCAGCCUGU |
| | GAACUGUGUUAAAUGAGGGU |
| | UAGUUUGACUGUAGCAAUAC |
| | AGUCUUGCUUUCUGACCCUA |
| | GUAGCUGCUCACCCUGAUGC |
| | UGCUGUCUUCGGACAGGAUA |
| | GGUGCGCUCCCAGCAAUAAG |
| | GGCGCGGAUGUACUGCUGUA |
| | GUGGCUACUGAAUCACCCCC |
| | GAUCAAGGGGGAACCCUAAA |
| | AGGGUUGAAAGNNNNNNNNN |
| | NNNNNNNNNNNNNN |
| Transposon left end | TGTACAGTGACTAATTATAT |
| (TE-L) (SEQ ID NO: 6) | GTCGTCGGTGACAAATTGTT |
| | GTCATTGAGCCAGACTAGTT |
| | GTCGTCGTGGCAAATTAGGT |
| | GTCGCTCATTTAATGGTGAC |
| | AAATTAATGTCGCTAAGATA |
| | ATACACTCTGTAATTATCAT |
| | ACAGAACAATTCAAACAAGC |
| | GGATAAAAGGACTTGCTTTC |
| | AACCCACCCCTAAATTTAAT |
| | ACTTACTGAAACCCAAAACT |
| | AATCTCAGTTTCAAATAT |
| Transposon right end | AATACATAAGTTTTTTATAA |
| (TE-R) (SEQ ID NO: 7) | ATACATATTGTTATTCTAGG |
| | TGTAGATAAAGCGACAGTCA |
| | ATTTGTCATTATGAAAATAC |
| | ACAAAAGCTTTTTTCTATTT |
| | AAAAAAGCGACAACTAATTT |
| | GTCACTATTACGGATAACGA |
| | CATTTATTTTGTCACCATGA |
| | AGAAGTGATCCTAATTTTGT |
| | GAAAACGCTATAAGATATAC |
| | TGTACAAGCATTTTAGCAAT |
| | GACATTAATTTGTCACGACG |
| | ACAAATAAAAAGTCACTGTA |
| | CA |

To test bacterial activity of the recombinant nucleic acid targeting system described herein, a plasmid comprising a test payload and transposon ends (referred to herein as “pDonor plasmid B2”) and a plasmid comprising a specified target sequence (referred to herein as “pTarget plasmid C2”) were also cloned. A schematic of pDonor plasmid B2 is shown in FIG. 1B, and the sequences of the left end and the right end are shown in Table 1. pTarget plasmid C2 was a low copy bacterial plasmid containing a specific target site matching the targeting sequence of the sgRNA in pEffector plasmid A2 and an upstream GGTT sequence (FIG. 1C). The target site was introduced into pTarget plasmid C2 and was synthesized as a synthetic DNA sequence having a specific target sequence flanked on either side by restriction enzyme sites for cloning into pTarget plasmid C2.

The target and sgRNA sequences were PCR amplified with two overlapping oligos and were used as the template DNA. The PCR amplicons were designed such that the sequence of interest was flanked on either side with two unique BsaI cut sites. The corresponding sites were present in the pEffector plasmid A2 and pTarget plasmid C2. The PCR amplicons and the associated pEffector plasmid A2 or pTarget plasmid C2 were then cut at the sites described herein and ligated together using standard molecular biology cloning techniques.

Ligated pEffector plasmid A2 and pTarget plasmid C2 were transformed into a chemically competent bacterial cell line by heat shock, plated onto LB-agar plates containing carbenicillin (antibiotic resistance marker for pEffector plasmid A2) or chloramphenicol (antibiotic resistance marker for pTarget plasmid C2), and incubated at 37° C. overnight. Individual colonies were then picked, grown for about 12-16 h in 2-5 mL of LB containing carbenicillin (pEffector) or chloramphenicol (pTarget), and miniprep-purified using a commercially available kit. Purified plasmids were sequence verified using Illumina sequencing.

The pEffector plasmid A2, pDonor plasmid B2, and pTarget plasmid C2 were normalized to 10 ng/μL, then 2 μL (20 ng) of each were combined in equal amounts then co-transformed in electrocompetent PIR1 E. coli (Thermo Fisher). After a 1 h outgrowth at 37° C. with shaking, the cells were plated on LB-agar bioassay plates containing kanamycin, carbenicillin, and chloramphenicol and incubated for 16 h at 37° C. The cells were then harvested from the plate, and the plasmid DNA was miniprep-purified.

Miniprep-purified plasmid DNA was normalized to approximately 1 ng/ul and prepared for sequencing using a Nextera XT DNA Library Preparation Kit (Illumina) following the associated Tagmentation and PCR protocols. Following PCR, samples were combined and purified by gel extraction using the QIAquick Gel Extraction Kit (Qiagen), selecting for fragments 350-500 bp long. Purified DNA was loaded onto a NextSeq 550 sequencer and sequenced using either the 2×75 paired-end protocol with a 150 Mid Kit (v2.5).

Sequencing reads were demultiplexed to create individual fastq files for each sample. The first 50 nucleotides of each paired-end read were aligned to the pDonor plasmid B2, pTarget plasmid C2, and pEffector plasmid A2 separately. Instances where the two paired-end reads aligned to separate pDonor plasmid B2 and pTarget plasmid C2, separately, represented possible transposition events, and these “trans reads” were tracked and analyzed. Instances where the reads align to the pDonor plasmid B2 and pEffector plasmid A2 were also tracked and analyzed as a negative control. The positions of the two ends were then plotted to determine if transposition was occurring in a targeted manner near the target site. The transposition events that were specific to the recombinant nucleic acid targeting system described herein were expected to map to the transposase ends and be located near the target sequence.

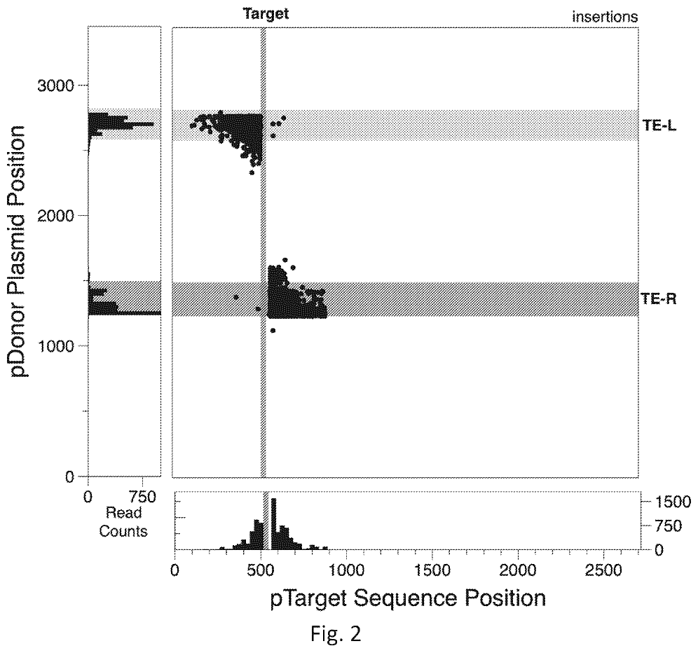

FIG. 2 shows the trans reads mapped for payload insertion events in pTarget. The x- and y-axes represent the alignment position to pTarget plasmid C2 and pDonor plasmid B2, respectively, where each point is a paired-end read where one end aligns to pDonor plasmid B2 and the other end aligns to pTarget plasmid C2. Histograms along the vertical and horizontal axes display the number of reads in one of the paired-end reads aligning to pDonor plasmid B2 or pTarget plasmid C2, respectively. The shaded regions denoted as ‘TE-L’ or ‘TE-R’ represent the transposon left end and transposon right end, respectively, which define the outer edges of the payload sequence (between sequence positions 1237-2821). The shaded region denoted as ‘target’ represents the sequence within pTarget plasmid C2 that is targeted for transposition.

As shown in FIG. 2, two clusters of points were found between the TE-L region on the y-axis and left of the target region on the x-axis (upstream) and within the TE-R region on y-axis and right of the target region. This indicated that the payload inserted in a defined orientation such that the final product was (in order): the target sequence, the transposon left end (TE-L), and ending with the transposon right end (TE-R).

To determine the integration efficiency of the system, the cis (both paired-end reads aligned to the same plasmid) and trans (paired-end reads aligned to separate plasmids) reads were filtered to include only those that aligned to the pTarget plasmid C2 within 400 nucleotides of the target sequence. The number of trans reads passing these filters was then counted and divided by the total number of reads fulfilling these conditions to provide the percent integration. In doing so, the percent integration by the recombinant nucleic acid targeting system described herein was found to be 65.6%+2.5%. Insertions occurred 40-60 bp downstream from the 5′ side of the target sequence. No insertion events into pEffector (the negative control), instead of pTarget, were observed.

This Example thus shows that the recombinant nucleic acid targeting system described herein was active in E. coli by inserting a defined payload sequence in a specific location with a specific orientation.

### Example 2—Analysis of Transposase Activity In Vitro

This example describes the in vitro verification of the minimal components required for the activity of the recombinant nucleic acid targeting system described herein.

Plasmids encoding each protein in the recombinant nucleic acid targeting system described herein with an N-terminal His-SUMO tag are designed and generated by multi-fragment Gibson Assembly. Each of the Cas12k, the TniA, the TniB, and the TniQ proteins, are placed directly downstream of a T7 promoter and provided a high copy origin of replication and an ampicillin resistance cassette for selection. Fragments for the Gibson Assembly reaction are generated by PCR of plasmids described in Example 1 or ordered as synthetic DNA from Integrated DNA Technologies (IDT). The assembled plasmid is then transformed into chemically competent E. coli cells and plated onto LB-Agar containing the carbenicillin. Single colonies are grown, miniprepped, and sequence verified as described in Example 1.

These plasmids are transformed into chemically competent E. coli cells and grown on LB-Agar plates with carbenicillin overnight to create fresh colonies. One or multiple colonies are then inoculated into LB containing carbenicillin and grown overnight at 37° C. in a shaking incubator. This starter culture is then diluted 1000-fold into 1 L of Terrific Broth and grown in a shaking incubator until an optical density between 0.4 and 1.0 is reached. Expression of the proteins of interest is induced by the addition of IPTG (200 nM to 1 uM final concentration), and cells are allowed to continue to grow at 18-20° C. with shaking overnight. Cells are then pelleted.

Cell pellets are resuspended in a buffer comprising 50 mM Tris-NaOH (pH7.4), 500 mM NaCl, 20 mM Imidazole, 14.3 mM 2-mercaptoethanol, 1 mM DTT, 5% Glycerol, and 1× dilution of cOmplete™ Protease Inhibitor Cocktail (Sigma) at 4° C. Cells are lysed and stored on ice. Cell debris is removed through two rounds of centrifugation at 18,000 rpm at 4° C. for 30 minutes followed by collection of the supernatant. The purified lysate is then purified by Fast Paced Liquid Chromatography (FPLC). Fractions containing the protein of interest are identified by polyacrylamide gel electrophoresis (PAGE) and pooled together.

Approximately 400 U of SUMO Protease 1 (LifeSensors or Lucigen) is combined with the pooled fractions (for cleavage of the N-terminal His-SUMO tag) and the sample is dialyzed overnight into 3 L of buffer comprising 50 mM Tris-NaOH (pH 7.4), 200 mM NaCl, 20 mM Imidazole, 14.3 mM 2-mercaptoethanol, 1 mM DTT, and 5% Glycerol using Slide-A-Lyzer™ G2 Dialysis Cassettes (Thermo Scientific) with the appropriate molecular weight cutoff at 4° C. The sample is then purified by FPLC, and the flow through is collected. Fractions containing the protein of interest are identified by PAGE and pooled together. The pooled fractions are then concentrated and purified by size-exclusion, and fractions containing the protein of interest are combined. Protein concentrations are determined by UV/Visible spectroscopy. The final buffer comprises 50 mM Tris-NaOH (pH 7.4), 200 mM NaCl, 14.3 mM 2-mercaptoethanol, 1 mM DTT, and 15% Glycerol. Protein extinction coefficients are calculated based on the primary sequence.

A DNA template encoding the sgRNA molecule downstream of a T7 RNA polymerase promoter is prepared by PCR amplification using NEBNext® High-Fidelity 2×PCR Master Mix (NEB). T7 transcription is performed using the HiScribe™ T7 High Yield RNA Synthesis Kit (NEB) following the NEB Standard RNA Synthesis protocol. Transcription reactions are allowed to proceed for 2-16 hrs at 37° C. The DNA template is removed by the addition of TURBO DNase Buffer (1× final concentration) and TURBO DNase (0.02-0.2 U/ul final concentration; ThermoFisher Scientific). DNase reactions are performed at 37° C. for 15-30 min. RNA is purified using the RNA Clean & Concentrator Kit-25 (ZymoResearch). The final RNA yield is determined by UV/Visible spectroscopy with a NanoDrop™ 2000c (ThermoFisher Scientific) or Qubit™ 3 Fluorometer (ThermoFisher Scientific) with the Qubit RNA HS Assay Kit (ThermoFisher Scientific). An extinction coefficient is estimated based on the RNA primary sequence.

Each of the purified of the Cas12k, the TniA, the TniB, and the TniQ proteins is diluted to a concentration of 2 μM in 1× protein dilution buffer (25 mM Tris pH 8, 500 mM NaCl, 1 mM EDTA, 1 mM DTT, 25% glycerol). In vitro integration assays are performed using each of the Cas12k, the TniA, the TniB, and the TniQ protein at a final concentration of 50 nM, 20 ng of pTarget, 100 ng of pDonor, and RNA at a final concentration of 600 nM in a reaction buffer (e.g., 26 mM HEPES pH 7.5, 4.2 mM Tris pH 8, 50 μg/mL BSA, 2 mM ATP, 2.1 mM DTT, 0.05 mM EDTA, 0.2 mM MgCl₂, 28 mM NaCl, 21 mM KCl, 1.35% glycerol, pH 7.5) supplemented with 15 mM MgOAc₂. Total reaction volumes are 20 μL, and reactions are incubated for 2 hours at 37° C.

Post incubation, the nucleic acids in the samples are purified using Agencourt AMPure XP beads and eluted in a final volume of 12 μL water. The concentration of DNA in the purified samples is quantified using a Quant iT Picogreen dsDNA assay kit (ThermoFisher). Following quantification, the DNA content in the samples is normalized such that the same amount of input DNA is used across all samples for subsequent analysis.

The normalized samples are then tested for integration with PCR using a set of two primers: one specific for pTarget and one specific for pDonor. The resulting PCR products are analyzed by agarose gel electrophoresis. PCR products of expected sizes for transposition are then further analyzed by Sanger sequencing to confirm transposition. The PCR template material is also analyzed using the unanchored Nextera method described in Example 1 to measure the level of integration. Additional control reactions are included to test programmability of integration in the: i) absence of Cas12k, ii) absence of RNA components, iii) pTarget lacking the correct target site, and iv) non-targeting RNA components.

This in vitro integration reaction can also be used to analyze different requirements of the recombinant nucleic acid targeting system described herein, for activity. One such experiment is to test different sequences for the RNA guide. Other experiments are performed to determine minimal requirements of the transposase ends within the payload sequence and the effect of payload size on transposition efficiency.

What is claimed is:

1. A recombinant nucleic acid comprising a first promoter operably linked to a first polynucleotide and a second promoter operably linked to a second polynucleotide,
wherein the first polynucleotide comprises:
a nucleic acid sequence encoding a TniA protein, or functional fragment thereof, a nucleic acid sequence encoding a TniB protein, or functional fragment thereof, and a nucleic acid sequence encoding a TniQ protein, or functional fragment thereof, and
a nucleic acid sequence encoding a CRISPR associated (Cas) protein, wherein the Cas protein comprises an amino acid sequence that is at least 95% identical to an amino acid sequence set forth in SEQ ID NO: 1;
wherein the second polynucleotide comprises:
a nucleic acid sequence encoding a guide RNA (gRNA), wherein the gRNA is capable of hybridizing with a target sequence.

2. The recombinant nucleic acid of claim 1, wherein the TniA protein comprises an amino acid sequence that is at least 95% identical to an amino acid sequence set forth in SEQ ID NO: 2.

3. The recombinant nucleic acid of claim 1, wherein the TniB protein comprises an amino acid sequence that is at least 95% identical to an amino acid sequence set forth in SEQ ID NO: 3.

4. The recombinant nucleic acid of claim 1, wherein the TniQ protein comprises an amino acid sequence that is at least 95% identical to an amino acid sequence set forth in SEQ ID NO: 4.

5. The recombinant nucleic acid of claim 1, wherein the TniA protein comprises an amino acid sequence that is at least 95% identical to an amino acid sequence set forth in SEQ ID NO: 2, the TniB protein comprises an amino acid sequence that is at least 95% identical to an amino acid sequence set forth in SEQ ID NO: 3, and the TniQ protein comprises an amino acid sequence that is at least 95% identical to an amino acid sequence set forth in SEQ ID NO: 4.

6. The recombinant nucleic acid of any one of claims 1-5, wherein the gRNA is capable of complexing with the Cas protein to form a gRNA-Cas protein complex.

7. The recombinant nucleic acid of any one of claims 1-6, wherein the gRNA comprises a CRISPR/Cas system associated RNA (crRNA) sequence.

8. The recombinant nucleic acid of any one of claims 1-7, wherein the gRNA is a single guide RNA further comprising a trans-activating CRISPR/Cas system RNA (tracrRNA) sequence.

9. The recombinant nucleic acid of any one of claims 1-8, wherein the gRNA comprises a nucleotide sequence as set forth in SEQ ID NO: 5.

10. A vector comprising the recombinant nucleic acid of any one of claims 1-9.

11. A bacterial cell comprising the vector of claim 10.

12. A recombinant nucleic acid targeting system for sequence-specific modification of a target sequence, the system comprising:
a TniA protein, a TniB protein, and a TniQ protein, or a polynucleotide encoding the TniA protein, the TniB protein, and the TniQ protein;
a Cas protein comprising an amino acid sequence that is at least 95% identical to an amino acid sequence set forth in SEQ ID NO: 1 or a polynucleotide encoding the Cas protein, wherein the Cas protein comprises an amino acid sequence that is at least 95% identical to an amino acid sequence set forth in SEQ ID NO: 1; and
a guide RNA (gRNA) or a polynucleotide encoding the gRNA,
wherein the gRNA is capable of complexing with the Cas protein to form a gRNA-Cas protein complex.

13. The recombinant nucleic acid targeting system of claim 12, wherein the TniA protein comprises an amino acid sequence that is at least 95% identical to an amino acid sequence set forth in SEQ ID NO: 2.

14. The recombinant nucleic acid targeting system of claim 12, wherein the TniB protein comprises an amino acid sequence that is at least 95% identical to an amino acid sequence set forth in SEQ ID NO: 3.

15. The recombinant nucleic acid targeting system of claim 12, wherein the TniQ protein comprises an amino acid sequence that is at least 95% identical to an amino acid sequence set forth in SEQ ID NO: 4.

16. The recombinant nucleic acid targeting system of claim 12, wherein the TniA protein comprises an amino acid sequence that is at least 95% identical to an amino acid set forth in SEQ ID NO: 2, the TniB protein comprises an amino acid sequence that is at least 95% identical to an amino acid set forth in SEQ ID NO: 3, and the TniQ protein comprises an amino acid sequence that is at least 95% identical to an amino acid set forth in SEQ ID NO: 4.

17. The recombinant nucleic acid targeting system of any one of claims 12-16, wherein the gRNA comprises a CRISPR/Cas system associated RNA (crRNA) sequence.

18. The recombinant nucleic acid targeting system of any one of claims 12-17, wherein the gRNA is a single guide RNA (sgRNA) further comprising a trans-activating CRISPR/Cas system RNA (tracrRNA) sequence.

19. The recombinant nucleic acid targeting system of any one of claims 12-18, wherein the gRNA comprises a nucleotide sequence as set forth in SEQ ID NO: 5.

20. The recombinant nucleic acid targeting system of any one of claims 12-19, further comprising a target polynucleotide, wherein the target polynucleotide comprises (i) a target sequence capable of hybridizing to the gRNA and (ii) a protospacer-adjacent motif (PAM) sequence.

21. The recombinant nucleic acid targeting system of claim 20, wherein the PAM sequence comprises a nucleotide sequence selected from the group consisting of nucleotide sequences as set forth in 5′-GTN-3′, 5′-NGTN-3′, 5′-GGTN-3′, 5′-GGTA-3′, 5′-GGTC-3′, 5′-GGTG-3′, 5′-GGTT-3′, 5′-GTT-3′, 5′-GTA-3′, 5′-GTC-3′, and 5′-GTG-3′.

22. The recombinant nucleic acid targeting system of claim 21, wherein the PAM sequence comprises a nucleotide sequence as set forth in 5′-GGTT-3′.

23. The recombinant nucleic acid targeting system of any one of claims 12-22, further comprising a donor polynucleotide, wherein the donor polynucleotide comprises a payload sequence for insertion into the target polynucleotide.

24. The recombinant nucleic acid targeting system of claim 23, wherein the donor polynucleotide further comprises a nucleic acid sequence encoding a transposon left end (TE-L) and a nucleic acid sequence encoding a transposon right end (TE-R).

25. The recombinant nucleic acid targeting system of claim 24, wherein the TE-L comprises a nucleic acid sequence that is at least 95% identical to an amino acid set forth in SEQ ID NO: 6.

26. The recombinant nucleic acid targeting system of claim 24 or 25, wherein the TE-R comprises a nucleic acid sequence that is at least 95% identical to an amino acid set forth in SEQ ID NO: 7.

27. A recombinant nucleic acid targeting system for sequence-specific modification of a target sequence, the system comprising:
a TniA protein comprising an amino acid sequence that is at least 95% identical to an amino acid sequence set forth in SEQ ID NO: 2; and
a donor polynucleotide, wherein the donor polynucleotide comprises
a payload sequence for insertion into the target sequence
a nucleic acid sequence encoding a transposon left end (TE-L) that is at least 95% identical to a nucleic acid sequence set forth in SEQ ID NO: 6, and
a nucleic acid sequence encoding a transposon right end (TE-R) that is at least 95% identical to a nucleic acid sequence set forth in SEQ ID NO: 7.

28. The recombinant nucleic acid targeting system of claim 27, further comprising a Type V-K Cas protein (e.g., a Cas12k protein).

29. The recombinant nucleic acid targeting system of claim 28, wherein the Cas protein comprises an amino acid sequence that is at least 95% identical to an amino acid sequence set forth in SEQ ID NO: 1 or a polynucleotide encoding the Cas protein, wherein the Cas protein comprises an amino acid sequence that is at least 95% identical to an amino acid sequence set forth in SEQ ID NO: 1.

30. The recombinant nucleic acid targeting system of any of claims 27-29, further comprising a guide RNA (gRNA) or a polynucleotide encoding the gRNA, wherein the gRNA is capable of complexing with the Cas protein to form a gRNA-Cas protein complex.

31. The recombinant nucleic acid targeting system of any one of claims 27-30, further comprising one or more of a TniB protein and a TniQ protein.

32. The recombinant nucleic acid targeting system of claim 31, wherein the TniB protein comprises an amino acid sequence that is at least 95% identical to an amino acid sequence set forth in SEQ ID NO: 3 or a polynucleotide encoding the TniB protein, wherein the TniB protein comprises an amino acid sequence that is at least 95% identical to an amino acid sequence set forth in SEQ ID NO: 3.

33. The recombinant nucleic acid targeting system of claim 31, wherein the TniQ protein comprises an amino acid sequence that is at least 95% identical to an amino acid sequence set forth in SEQ ID NO: 4 or a polynucleotide encoding the TniQ protein, wherein the TniQ protein comprises an amino acid sequence that is at least 95% identical to an amino acid sequence set forth in SEQ ID NO: 4.

34. The recombinant nucleic acid targeting system of any one of claims 12-33, wherein at least one of the Cas protein, the TniA protein, the TniB protein, and the TniQ protein is purified protein.

35. A bacterial cell comprising the recombinant nucleic acid targeting system of any one of claims 12-34.

36. A method for modifying a target polynucleotide in a bacterial cell, said method comprising introducing into to the cell:
(i) a first recombinant nucleic acid comprising:
a polynucleotide encoding a TniA protein, or functional fragment thereof, a polynucleotide encoding a TniB protein, or functional fragment thereof, and a polynucleotide encoding a TniQ protein, or functional fragment thereof;
a polynucleotide encoding a Cas protein, wherein the Cas protein comprises an amino acid sequence as set forth in SEQ ID NO: 1; and
a polynucleotide encoding a guide RNA (gRNA), wherein the gRNA is capable of complexing with the Cas protein to form a gRNA-Cas protein complex;
(ii) a second recombinant nucleic acid comprising a target polynucleotide, wherein the target polynucleotide comprises (a) a target sequence capable of hybridizing to the gRNA and (b) a PAM sequence; and
(iii) a third recombinant nucleic acid comprising a donor polynucleotide, wherein the donor polynucleotide comprises a payload sequence for insertion into the target polynucleotide, thereby modifying the target polynucleotide.

37. The method of claim 36, wherein the donor polynucleotide further comprises a nucleic acid sequence encoding a transposon left end (TE-L) and a nucleic acid sequence encoding a transposon right end (TE-R).

38. The method of any one of claim 36 or 37, wherein the TniA protein comprises an amino acid sequence that is at least 95% identical to an amino acid sequence as set forth in SEQ ID NO: 2.

39. The method of any one of claim 36 or 37, wherein the TniB protein comprises an amino acid sequence that is at least 95% identical to an amino acid sequence as set forth in SEQ ID NO: 3.

40. The method of any one of claim 36 or 37, wherein the TniQ protein comprises an amino acid sequence that is at least 95% identical to an amino acid sequence as set forth in SEQ ID NO: 4.

41. The method of any one of claim 36 or 37, wherein the TniA comprises an amino acid sequence that is at least 95% identical to an amino acid sequence set forth in SEQ ID NO: 2, the TniB comprises an amino acid sequence that is at least 95% identical to an amino acid sequence set forth in SEQ ID NO: 3, and the TniQ comprises an amino acid sequence that is at least 95% identical to an amino acid sequence set forth in SEQ ID NO: 4.

42. The method of any one of claims 36-41, wherein the PAM sequence comprises a nucleotide sequence selected from the group consisting of nucleotide sequences as set forth in 5′-GTN-3′, 5′-NGTN-3′, 5′-GGTN-3′, 5′-GGTA-3′, 5′-GGTC-3′, 5′-GGTG-3′, 5′-GGTT-3′, 5′-GTT-3′, 5′-GTA-3′, 5′-GTC-3′, and 5′-GTG-3′.

43. The method of claim 42, wherein the PAM sequence comprises a nucleotide sequence as set forth in 5′-GGTT-3′.

44. The method of any one of claims 37-43, wherein the TE-L has a nucleic acid sequence as set forth in SEQ ID NO: 6.

45. The method of any one of claims 37-43, wherein the TE-R has a nucleic acid sequence as set forth in SEQ ID NO: 7.

46. The method of any one of claims 37-45, wherein the bacterial cell is Escherichia coli.
